(19) United States
(12) Patent Application Publication
(10) Pub. No.: US 2026/0117292 A1
(43) Pub. Date: Apr. 30, 2026

(54) SYSTEMS AND METHODS FOR MULTIPLEXED POLYMERASE CHAIN REACTION PROCESSES AND DATA ANALYSIS

(71) Applicant: Life Technologies Corporation, Carlsbad, CA (US)

(72) Inventors: Noah Elder, Lafayette, CA (US); Carmen Gjerstad, Millbrae, CA (US); Harrison Leong, San Francisco, CA (US); Mark Shannon, San Francisco, CA (US)

(21) Appl. No.: 19/380,075

(22) Filed: Nov. 5, 2025

Related U.S. Application Data
(63) Continuation of application No. PCT/US2024/029076, filed on May 13, 2024.
(60) Provisional application No. 63/501,880, filed on May 12, 2023.

(51) Int. Cl. C12Q 1/6851

(52) U.S. Cl. CPC C12Q 1/6851

(57) ABSTRACT

Systems and methods that enable analyte detection in a multiplexed amplification process can include obtaining, at multiple time points during the amplification process, composite fluorescence signal data associated with a composite fluorescence signal from at least a first probe type comprising a first fluorophore and a second probe type comprising a second fluorophore which has substantially overlapping spectral characteristics as said first fluorophore, the first probe type and the second probe type differing in thermal and/or temporal properties; and determining, based at least partially on the composite fluorescence signal data, fluorescence signal data associated with a fluorescence signal from a given probe type of the first probe type or the second probe type during the amplification process.

### CROSS-REFERENCE

This application is a bypass continuation of International Application No. PCT/US2024/029076 filed on May 13, 2024, which claims priority to U.S. Provisional Application No. 63/501,880, filed May 12, 2023. The entire contents of each of the foregoing applications are hereby incorporated by reference in their entirety.

### INTRODUCTION

Nucleic acid detection assays are often carried out by adding a sample that is suspected of including one or more target nucleic acids to a reaction mixture. The reaction mixture can include one or more detectable labels each designed to associate with a different target nucleic acid and generate a signal that corresponds to the amount of the associated target nucleic acid in the reaction mixture. In a “singleplex” assay, the reaction mixture includes a single detectable label designed to associate with a single target nucleic acid. Conversely, in a “multiplex” assay, the reaction mixture includes multiple, different detectable labels each typically designed to be specific to a different target nucleic acid.

Multiplex assays are therefore capable of detecting multiple different targets in a single reaction mixture. In some applications, the detectable labels are fluorescent dyes integrated with a nucleic acid probe, a primer, or some other nucleic acid molecule designed to specifically hybridize with the corresponding target nucleic acid with which it is designed to associate.

Challenges can arise when implementing multiplex systems and processes for determining the relative amounts of different target nucleic acids in a sample. In particular, using detectable labels that have overlapping emission spectra can be challenging to determine the respective contributions of each label individually and thus of the respective different target nucleic acids with which they are associated.

A need exists to provide more robust systems and methods for carrying out multiplex nucleic acid detection assays, such as nucleic acid detection utilizing various polymerase chain reaction (PCR) assays for example.

### BRIEF DESCRIPTION OF THE DRAWINGS

Various objects, features, characteristics, and advantages of the inventions within the scope of the present disclosure will become apparent and more readily appreciated from the following description of various embodiments, taken in conjunction with the accompanying drawings and the appended claims, all of which form a part of this specification. In the Drawings, like reference numerals may be utilized to designate corresponding or similar parts in the various Figures, and the various elements depicted are not necessarily drawn to scale, wherein:

FIG. 1 illustrates emission spectra for various fluorescent dyes that can be used in nucleic acid detection assays;

FIG. 2A is a schematic overview of a technique for enabling detection of multiple target nucleic acids within the same detection channel, according to various embodiments of the present disclosure;

FIG. 2B is a graph showing signal response over time for the technique outlined in FIG. 2A, according to various embodiments of the present disclosure;

FIG. 3A illustrates activity of a cleavable probe and a non-cleavable probe during annealing, extension, and denaturation steps of a thermal cycle, according to embodiments of the present disclosure;

FIG. 3B is a graph showing fluorescent signal response over time during thermal cycling of an amplification process that utilizes the cleavable and non-cleavable probes of FIG. 3A, according to embodiments of the present disclosure;

FIG. 4 illustrates a flow diagram depicting exemplary actions associated with enabling analyte detection in a multiplexed amplification process, according to embodiments of the present disclosure;

FIGS. 5A and 5B illustrate results of a duplex assay test in which TaqMan probes and extendable fluorogenic (EF) probes were designed to generate fluorescence signals in the same dye channel (FIG. 5A) or in different dye channels (FIG. 5B);

FIG. 5C compares the EF-associated fluorescence signal as derived using the results of the assay of FIG. 5A with the EF-associated fluorescence signal as directly measured in the assay of FIG. 5B.

FIG. 5D illustrates fluorescent signals of a TaqMan probe and an extendable fluorogenic probe (EF) at extension and denaturation stages;

FIG. 6 illustrates the results of another 9-plex assay test that included 5 different detection channels/dyes, four detection channels with a corresponding TaqMan probe and an EF probe (each channel having a differing dye common to the TaqMan and EF probes in that channel) and one channel with only an TaqMan probe, according to embodiments of the present disclosure;

FIG. 7 illustrates a flow diagram depicting exemplary actions associated with enabling analyte detection in a multiplexed dPCR assay, according to embodiments of the present disclosure;

FIG. 8 illustrates various data visualizations and corresponding dPCR data analysis techniques in accordance with embodiments of the present disclosure;

FIG. 9 illustrates various data visualizations and corresponding dPCR data analysis techniques in accordance with yet other embodiments of the present disclosure;

FIG. 10A illustrates a process of using a tailed primer, specific to a nucleic acid target, to form a template to which the EF probe can hybridize; and

FIG. 10B illustrates an example tailed forward primer, reverse primer, and EF probe that may be included in the reaction mixture to implement the process of FIG. 10A.

### DETAILED DESCRIPTION

In the context of a nucleic acid probe and a target nucleic acid, the term “specifically interact” (and similar terms) indicates that the probe is designed to interact with the target to a greater degree than with non-target nucleic acids also present in the reaction mixture. For example, specific interaction may include hybridization of the probe, in whole or in part, with the corresponding target. The hybridization between the probe and target need not be 100%. For example, functionally effective interaction may be accomplished with probes having homology to their respective target of at least about 70%, at least about 75%, at least about 80%, at least about 85%, at least about 90%, at least about 95%, at least about 97%, at least about 98%, at least about 99%, or up to 100%.

As used herein, a “detection channel” is a specified, contiguous subset of the total range of possible values of detectable signals. For example, where the detectable signals are fluorescence signals, a detection channel (e.g., fluorescence channel or dye channel) can represent a wavelength band of specified size. A detection channel may, for example, have a band size of about 10-60 nm, depending on instrument sensitivity and/or desired signal granularity. A detection channel may additionally or alternatively be defined according to the optical filter arrangement used to measure the detectable signals. Each different detection channel typically comprises a specific optical filter arrangement to block non-channel emissions. Thus, as a functional definition, each detectable signal within a given optical filter arrangement may be considered as being within the same detection channel.

As used herein, separate fluorescence signals that have “substantially identical fluorescence” provide fluorescence emissions within similar wavelength bands. For example, a first fluorescence signal and a second fluorescence signal with substantially identical fluorescence may have emission peaks that differ by no more than about 10 nm, or no more than about 8 nm, or no more than about 6 nm, or no more than about 4 nm, or no more than about 2 nm, or no more than about 1 nm, or that are substantially indistinguishable from one another based on the sensitivity of the detection instrument used to measure the fluorescence emissions. Additionally, or alternatively, fluorescence signals may be considered to have “substantially identical fluorescence” in applications where they are measured using the same optical filter arrangement.

As used herein, a “substantial signal” and/or a detectable signal that has “substantial fluorescence” is a signal significantly above a background (i.e., baseline) level, including a fluorescence signal that is significantly above a background/baseline level of fluorescence. This may be defined by a threshold value that separates background fluorescence from substantial fluorescence. The threshold value may vary according to particular testing protocols and application needs. In some embodiments (without a passive reference), the threshold is set at a ARn of about 1,000 to about 30,000, or more commonly about 2,000 to about 20,000, or about 3,000 to about 15,000 or about 4,000 to about 6,000, for example, or within a range having endpoints defined by any two of the foregoing values. In some embodiments (e.g., with a passive reference), the threshold is set at a ARn of about 0.01 to 0.5, for example. In some embodiments, the threshold value is some percentage above the baseline level, such as about 5 percent to about 10 percent above the baseline level.

A “background” or “baseline” level of signal (i.e., background/baseline level of fluorescence) during an amplification process may be determined according to methods known to those of skill in the art. As a non-limiting example, the baseline level may be determined as the median signal of the amplification cycles before exponential amplification occurs. For example, exponential amplification may be determined when the change in signal from one amplification cycle to the next exceeds a certain percentage indicative of exponential change.

As a corollary, a signal and/or fluorescence level that is not “substantial” according to the foregoing may be described herein as “negligible.” Similarly, with respect to probe binding, a probe is “substantially bound” to its target when it is bound significantly above background (e.g., above binding to a non-target). Optionally, at least 1%, 5%, 10%, 20%, 50% or 80% of the probe or the target is bound.

As used herein, a “cleavable” probe is a probe that is intended to be cleaved as a result of specific interaction of the probe with its respective target, and to cause a release of the corresponding label and an increase in the corresponding detectable signal as a result. As used herein, a “non-cleavable” probe is a probe with a label that is intended to remain associated with the probe throughout the assay. In a non-cleavable probe, the corresponding detectable signal varies according to configuration changes of the probe rather than by release of the label from the probe.

The terms “detectable signal” and “label signal” are used synonymously herein. For example, a “first label signal” is the signal emitted by a first label of a first probe type and a “second label signal” is the signal emitted by a second label of a second probe type. A “total signal” is the total measured signal within a particular detection channel at a given time point or measurement point. Multiple different “detectable signals”/“label signals” may contribute to the same “total signal.” For example, a total signal may include signal generated by a first label of a first probe type and signal generated by a second label of a second probe type. In this regard, in some instances, a “total signal” may be regarded as a “composite signal.” In some embodiments, the signals are fluorescence signals, and terms such as “first fluorescence signal,” “second fluorescence signal,” and “total fluorescence signal” may be used as specific examples of the corresponding broader terms.

The terms “determine,” “calculate,” and “estimate” are used synonymously herein. These terms are not intended to imply an exact level of measurement precision. Thus, where a value is “determined,” “calculated,” or “estimated” using the embodiments described herein, it will be understood that such a value may include some degree of inherent error due to factors such as detection instrument tolerances, rounding, chemical reaction variability, and other inherent measurement imperfections known and understood by those of skill in the art.

For any given element of component of a described embodiment, any of the possible alternatives listed for that element or component may generally be used individually or in combination with one another, unless implicitly or explicitly stated otherwise.

In addition, unless otherwise indicated, numbers expressing quantities, constituents, distances, or other measurements used in the specification and claims are to be understood as optionally being modified by the term “about” or its synonyms. When the terms “about,” “approximately,” “substantially,” or the like are used in conjunction with a stated amount, value, or condition, it may be taken to mean an amount, value or condition that deviates by less than 20%, less than 10%, less than 5%, less than 1%, less than 0.1%, or less than 0.01% of the stated amount, value, or condition. At the very least, and not as an attempt to limit the application of the doctrine of equivalents to the scope of the claims, each numerical parameter should be construed in light of the number of reported significant digits and by applying ordinary rounding techniques.

Any headings and subheadings used herein are for organizational purposes only and are not meant to be used to limit the scope of the description or the claims.

It will also be noted that, as used in this specification and the appended claims, the singular forms “a,” “an” and “the” do not exclude plural referents unless the context clearly dictates otherwise. Thus, for example, an embodiment referencing a singular referent (e.g., “widget”) may also include two or more such referents.

It will also be appreciated that embodiments described herein may also include properties and/or features (e.g., ingredients, components, members, elements, parts, and/or portions) described in one or more separate embodiments and are not necessarily limited strictly to the features expressly described for that particular embodiment. Accordingly, the various features of a given embodiment can be combined with and/or incorporated into other embodiments of the present disclosure. Thus, disclosure of certain features relative to a specific embodiment of the present disclosure should not be construed as limiting application or inclusion of said features to the specific embodiment. Rather, it will be appreciated that other embodiments can also include such features.

### Overview of Intra-Channel Multiplexing

In various multiplex nucleic acid detection assays, each detectable label is assigned to a different target. The presence and/or amount of each target can then be determined by measuring the signal emitted from a detectable label in separate “detection channels” each corresponding to a specific property of the corresponding emitted signal. For example, in the context of fluorescence-emitting dyes as a detectable label, the separate detection channels can correspond to the emission wavelength spectrum associated with each dye. However, there can be some amount of overlap in the emission spectra of the different dyes. Increased overlap in emission spectra increases the difficulty in resolving the separate detected fluorescence emission signals and thus increases the difficulty in detecting and/or quantifying the respective targets.

While multiplexed dyes can be selected with the intent to minimize spectral overlap, the finite nature of the emission spectrum places practical limits on the number of separate dyes that can be combined in the same multiplex assay. As a result, at present, there are significant limitations to the number of different targets that can be detected and/or measured in a multiplex assay. Accordingly, there is an ongoing need for compositions and methods capable of increasing the “plexy” of detection assays. Moreover, it may be desirable to otherwise use dyes that have some degree of overlap in emission spectra and/or to use the same dye for different target nucleic acids.

Disclosed herein are systems and methods for enabling multiplexed nucleic acid detection assays that rely on polymerase chain reaction (PCR) processes by enabling determination of separate detectable signals, each associated with a different assay target nucleic acid, within the same detection channel (e.g., within a channel sensitive to emission (e.g, fluorescence emission) within a defined spectral range).

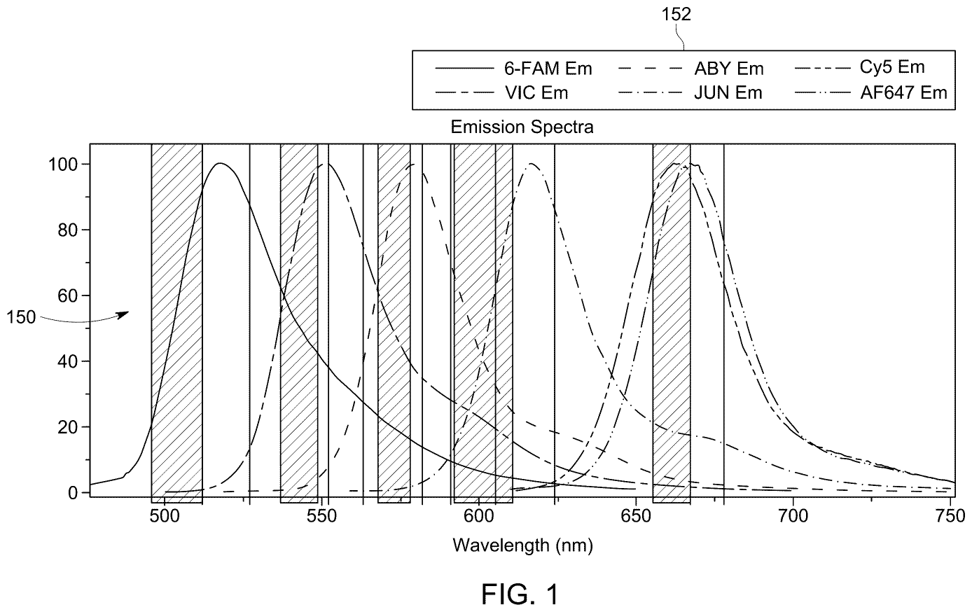

FIG. 1 illustrates emission spectra 150 for various fluorescent dyes 152 which can be used in nucleic acid detection assays. As discussed above, multiplex assays can assign each dye as a label for a separate target nucleic, and then determine the presence and/or amount of each target by measuring the fluorescence signal in separate detection channels each corresponding to differing emission wavelengths of the corresponding dye. As shown, there can be some (e.g., a substantial amount) of overlap in the emission spectra 150 of one or more of the dyes 152. For example, as illustrated in FIG. 1, the AF647 and Cy5 dyes have nearly the same emission spectra While multiplexed dyes are typically selected with the intent to minimize spectral overlap, the finite nature of the emission spectra places practical limits on the number of separate dyes that can be combined in the same multiplex assay, and therefore serves as a practical limit on the number of different targets that can be detected and/or measured.

Various embodiments described herein solve one or more of the foregoing problems by enabling analyte detection in multiplexed amplification processes by utilizing multiple detectable signals, each associated with a different assay target or set of targets, that have emission spectra corresponding to the same detection channel. The multiple detectable signals can be separately resolved and independently analyzed to thereby allow detection and/or quantification of each target. By allowing multiple targets/analytes to be assayed within the same detection channel, the disclosed embodiments can beneficially increase the “plexy” (i.e., number of targets that can be detected and quantified in a multiplex assay) without relying on additional dyes, dye channels, and concomitant issues of spectral overlap. Similarly, embodiments described herein can beneficially decrease the number of separate dyes required in a multiplex assay without lowering the plexy of the assay. In addition, various embodiments can allow for the same dye to be used as a label for different target nucleic acids in a multiplex assay, including to use the same dye for different targets detected in the same detection channel and at the same time during the reaction.

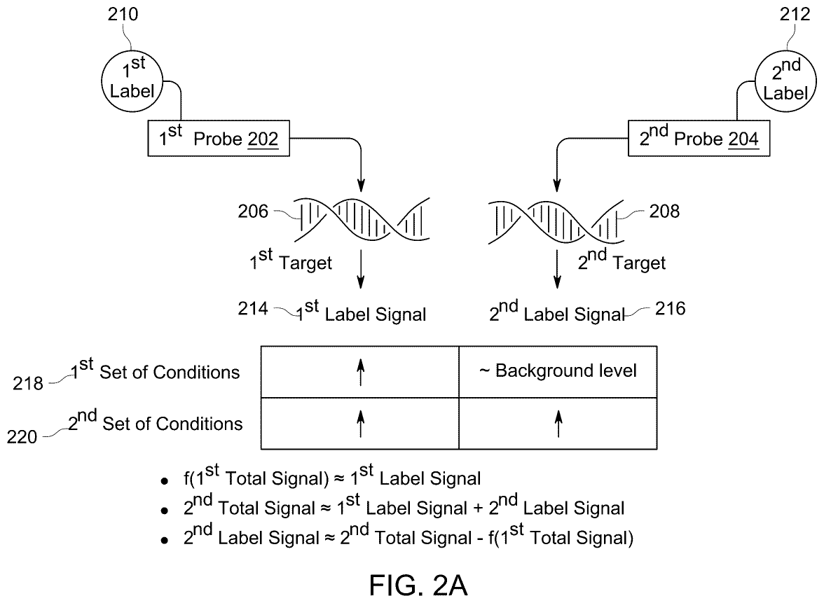

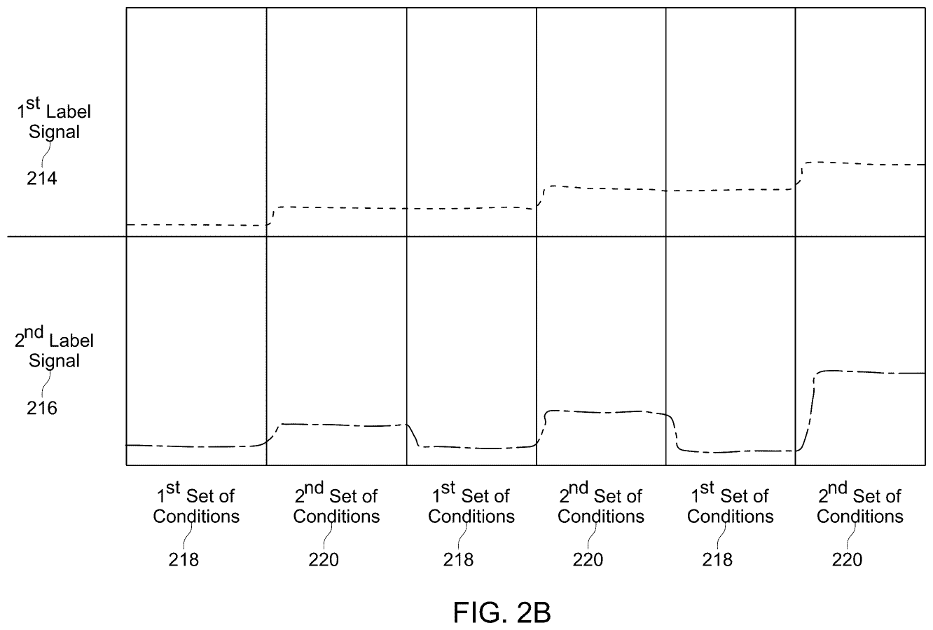

FIG. 2A is a schematic overview of a technique for enabling detection of multiple target nucleic acids within the same detection channel by providing different first and second probe types, varying the reaction mixture conditions, and measuring the resulting total signal at each set of conditions. As shown, a first probe 202 is designed to specifically interact with a first target 206. The first probe 202 includes a first label 210 that can generate a first label signal 214. A second probe 204 is designed to specifically interact with a second target 208 that is different from the first target 206. The second probe 204 includes a second label 212 that can generate a second label signal 216.

In some embodiments, the first and second labels 210 and 212 are the same. For example, the first and second labels 210 and 212 may comprise the same fluorescent dye. In some embodiments, the first and second labels 210 and 212 may be different, but are nonetheless designed to generate a substantially identical signal (e.g., substantially identical emission spectra). For example, the first and second labels 210 and 212 may comprise dyes that are chemically distinct yet function to emit fluorescence signals with similar wavelengths. In some embodiments, the first and second label signals 214 and 216 are measured using the same detection channel (e.g., including an optical filter arrangement) in the detection instrument.

The first probe 202 and second probe 204 may be provided in the same reaction mixture and allowed to specifically interact with any first and second target 206, 208, respectively, in the reaction mixture. As shown, the reaction mixture is subjected to at least two different sets of reaction conditions. The first probe 202 is designed such that the first label 210 generates the first label signal 214, to a degree proportional to the amount of specific interaction between the first probe 202 and first target 206, during both the first and second sets of conditions 218 and 220. In contrast, the second probe 204 is designed such that the second label 212 generates the second label signal 216, to a degree proportional to the amount of specific interaction between the second probe 204 and second target 208, during the second set of conditions 220 but not during the first set of conditions 218. In other words, under the first set of conditions 218, the first label signal 214 is increased as a result of specific interaction of the first probe 202 with the first target 206, but the second label signal 216 is not emitted as a result of specific interaction of the second probe 204 with the second target 208. Under the second set of conditions 220, the second label signal 216 is increased as a result of specific interaction of the second probe 204 with the second target 208, while the first label signal 214 also is further increased or remains at the increased level to at least some degree from the first set of conditions.

During the first set of conditions 218, the second label 212 will not generate “substantial fluorescence,” and the second label signal 216 will therefore not be substantially different from a background (i.e., baseline) level of fluorescence in the reaction mixture. That is, while there may be some non-zero level of signal generated by the second label 212 during the first set of conditions 218, the second label signal 216 will typically remain below a threshold value that separates background fluorescence from meaningful signal. This threshold may vary according to particular testing protocols and application needs, as discussed above.

In at least some embodiments, when both the first and the second targets 206 and 208 are present in the reaction mixture, the second label signal 216 will differ between the first and second sets of conditions 218 and 220 to a greater degree than the first label signal 214 will differ between the first and second sets of conditions 218 and 220. Thus, while the first label signal 214 may differ somewhat between the first and second sets of conditions 218 and 220, this difference will typically be less than the difference in the second label signal 216 between the first and second sets of conditions 218 and 220.

Various embodiments of the present disclosure exploit the difference in the way the first and second label signals 214 and 216 respond to the different sets of conditions so as to enable the detected first and second label signals 214 and 216 to be resolved (separated), even though detected within the same detection channel. For a given detection channel (e.g., for a given optical filter arrangement), the total signal (or composite signal) during the first set of conditions 218 (“the first total signal”) is measured, and the total signal during the second set of conditions 220 (“the second total signal”) is measured. Fluorescence signal data representing the first total signal is sometimes referred to herein as “first fluorescence signal data”, and fluorescence signal data representing the second total signal is sometimes referred to herein as “second fluorescence signal data” or “composite fluorescence signal data”. As used herein, first and second in this context is not necessarily used to denote a temporal order of detection or the conditions, although such temporal order may occur.

During the first set of conditions 218, the total signal will be substantially equal to the first label signal 214. That is, the first total signal is primarily composed of the first label signal 214, whereas contribution from the second label signal 216 is negligible. During the second set of conditions 220, the total signal will include a combination of the first and second label signals 214 and 216. The first and second label signals 214 and 216 can therefore be separately resolved based on the first and second total signals. For example, the first label signal 214 can be determined based on the first total signal, and the second label signal 216 can be resolved by subtracting the first total signal from the second total signal.

In some embodiments, the first label signal 214 is equated directly to the first total signal. In other embodiments, the first label signal 214 is determined as a function of the first total signal. In some embodiments, this function is a linear function (though non-linear functions may be used in some implementations). For example, as discussed above, the first label signal 214 may differ slightly between the first and second sets of conditions 218 and 220 even when the amount of first target 206 has not changed. In certain applications, the first label signal 214 under the second set of conditions 220 may better correspond to standard curves that equate the first label signal 214 to first target 206 amounts. Estimating the first label signal 214 as a function of the first total signal, rather than as directly equal to the first total signal, can therefore bring the calculated first label signal 214 closer to what would be measured under the second set of conditions 220 (i.e., without any interfering second label signal 216).

In some embodiments, the function for converting the first total signal to the first label signal 214 is determined by comparing, in the absence of any second probe interacting with a second target, the first label signal 214 under the first set of conditions 218 to the first label signal 214 under the second set of conditions 220. The first label signal 214 under the first set of conditions 218 and under the second set of conditions 220 can be correlated to one another according to a linear function. In other embodiments, they can be correlated using non-linear functions. When a linear function is used, a multiplier factor (e.g., correction factor) can be used to convert the first total signal to the first label signal 214. Once such a linear function is determined, it can be used in subsequent assays without necessarily requiring additional comparisons of the first label signal 214 under the first set of conditions 218 and under the second set of conditions 220 in the absence of the second probe with the second target. As noted above, in some embodiments, the function for converting the first total signal to the first label signal may be non-linear.

As described in greater detail below, the first probe 202 and the second probe 204 have different mechanisms of action that enable different signal responses, depending on the probe type, to the first and second sets of conditions 218 and 220. Beneficially, the ability to resolve the separate signals respectively associated with each of the different probe types can rely on attributes other than different melting temperatures of the probes. Thus, although the first probe 202 and second probe 204 may have dissimilar melting temperatures, such dissimilar melting temperatures is not a prerequisite to allow their associated label signals to be effectively resolved. In some embodiments, for example, a melting temperature (Tm) of the first probe 202 and a Tm of the second probe 204 are within about 8° C., or about 6° C., or about 4° C., or about 2° C. of each other, although such melting temperature differences are not limiting of the scope of the present disclosure. Moreover, in view of the techniques to enable differentiation in signal response in accordance with aspects of the present disclosure, a melting stage of an amplification process need not be relied on.

FIG. 2B is a graph schematically showing signal response over time for the technique outlined in FIG. 2A based on cycling of the reaction mixture between the first set of reaction conditions 218 and the second set of reaction conditions 220 and based on having both the first and second targets 206 and 208 present in the reaction mixture. The cycling of conditions may comprise, for example, the differing conditions of various stages associated with thermal cycling in a nucleic acid amplification reaction such as PCR for example. Under such a reaction, the first set of reaction conditions 218 correspond to supporting a denaturation stage of the thermal cycling and the second set of reaction conditions 220 correspond to supporting an annealing and/or extension stage (“annealing/extension stage”) of the thermal cycling. Thus, in various embodiments, the first set of reaction condition 218 include a first temperature or range of temperatures and the second set of reaction conditions include to a second temperature of range of temperatures.

As shown, both the first label signal 214 and the second label signal 216 increase under the second set of reaction conditions 220. Under the first set of reaction conditions 218, the first label signal 214 remains roughly the same as at the end of the previous cycle (though it may vary slightly, as discussed above), whereas the second label signal 216 drops to a level similar to the baseline signal level of the second label signal 216, which baseline signal level can be substantially constant over multiple amplification cycles. In other words, the second label signal 216 exhibits a baseline signal above the background signal level during the first set of reaction conditions. In some cases, the second label signal can exhibit a base line signal level that changes at differing stages of an amplification cycle, but nevertheless is sufficiently distinguishable from and lower than the level under the second set of reaction conditions. This may be due to a different state of the probe and proximity of a quencher to the label.

As shown, both the first label signal 214 and the second label signal 216 cumulatively increase at each successive occurrence of the second set of conditions 220. This is a result of additional specific interaction in the reaction mixture between the first probe 202 and the first target 206 and additional specific interaction in the reaction mixture between the second probe 204 and the second target 208. However, where the first label signal 214 remains at a similar level when moving from the end of one cycle to the beginning of another (i.e., when moving from the second set of conditions 220 at the end of a cycle to the first set of conditions 218 at the beginning of a subsequent cycle), the second label signal 216 returns to a level near baseline at the beginning of each cycle (i.e., at each occurrence of the first set of conditions 218).

While various embodiments described herein primarily focus on intra-channel multiplexing, the disclosed intra-channel multiplexing may be combined with inter-channel multiplexing to further increase the plexy of the assay. For example, an assay may be designed with multiple different dyes (and thus with multiple different detection channels), where two or more of the different channels each are configured to detect multiple detectable signals of signal responses from different targets which can be resolved in accordance with the techniques described herein.

### Cleavable & Non-Cleavable Probes

In some embodiments, the first probe (e.g., first probe 202) is a “cleavable” probe. The first probe may be designed such that the first label (e.g., first label 210) is detached from the first probe (and released from a corresponding quencher, for example) as a result of hybridization of the first probe to the first target (e.g., first target 206). Once released, the first label therefore continues to contribute to the total signal in the reaction mixture. The first probe may be a TaqMan probe, for example, which undergoes cleavage as a result of 5′ to 3′ exonuclease activity of DNA polymerase during extension of the target molecule to which the probe is hybridized. TaqMan probes are described in U.S. Pat. Nos. 4,889,818; 5,079,352; 5,210,015; 5,436,134; 5,487,972; 5,658,751; 5,210,015; 5,487,972; 5,538,848; 5,618,711; 5,677,152; 5,723,591; 5,773,258; 5,789,224; 5,801,155; 5,804,375; 5,876,930; 5,994,056; 6,030,787; 6,084,102; 6,127,155; 6,171,785; 6,214,979; 6,258,569; 6,814,934; 6,821,727; 7,141,377; and 7,445,900, all of which are hereby incorporated herein by reference.

In some embodiments, the second probe (e.g., second probe 204) is a “non-cleavable” probe. The label of a non-cleavable probe is intended to remain associated with the probe throughout the assay, and to vary in the level of generated signal according to probe configuration rather than release of the label. The second probe may be an extendable fluorogenic probe (EF), for example, which quenches the label when in a single stranded configuration but allows signal when incorporated into a double stranded molecule.

EF probes can be, for example, a universal extendable fluorogenic probe or an extendable hairpin probe designed for specific target amplification.

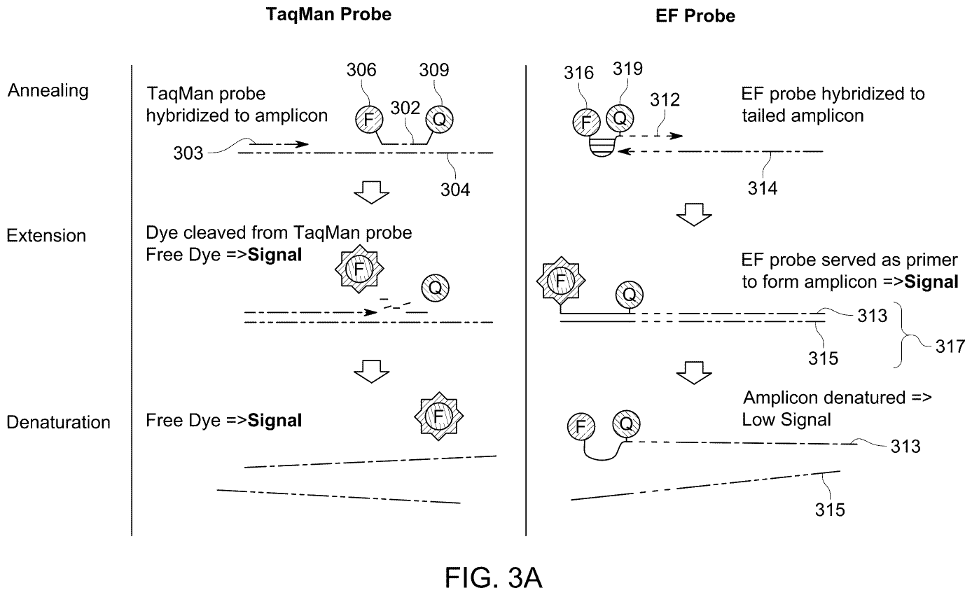

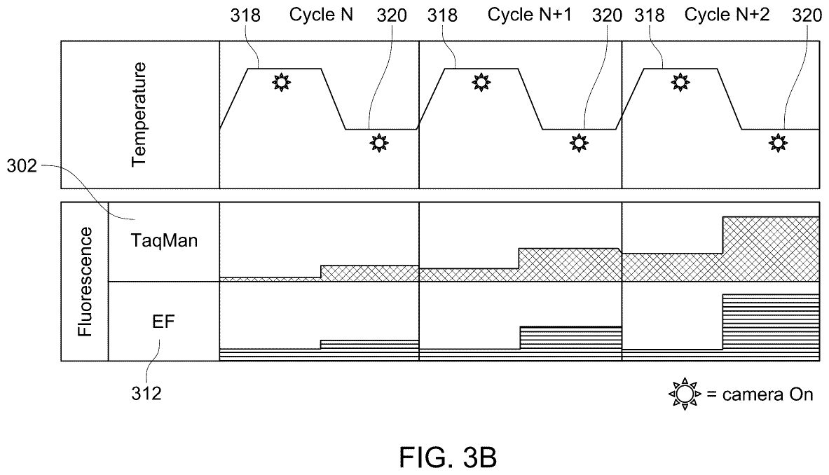

FIG. 3A illustrates activity of a cleavable probe 302, which in various embodiments can be a TaqMan probe and a non-cleavable probe 312, which in various embodiments can be a EF probe, during annealing, extension, and denaturation stages of a PCR reaction thermal cycle. As shown, the TaqMan probe 302 hybridizes to its corresponding target nucleic acid amplicon 304 (as used herein target nucleic acid amplicon can refer to a single strand of the target double-stranded nucleic acid and should be understood by reference to the context when describing a PCR reaction) during the annealing stage. During extension of a primer 303 hybridized to the target nucleic acid amplicon 304 upstream of the probe 302, the 5′ to 3′ exonuclease activity of a DNA polymerase cleaves the TaqMan probe label 306 from the remainder of the probe 302, thereby separating it from the corresponding TaqMan probe quencher 309. This leads to a corresponding increase in the fluorescence signal. During denaturation, the label 306 remains free within the reaction mixture solution and thus continues to contribute to the total fluorescence signal.

The EF 312 includes a EF label 316 and a EF quencher 317 which remain in proximity to one another while the probe 312 is in a single stranded configuration. The fluorescence signal from the label 316 thus remains substantially quenched while the EF is in a single stranded configuration. During the annealing and extension stages, the EF 312 hybridizes to its corresponding target template amplicon 314 and is extended to form an extended probe amplicon 313. Extension of target template 314 then forms the complement 315 of the extended probe amplicon 113. The resulting double stranded amplicon 317 forces the label 316 away from the quencher 318 to a distance sufficient to allow fluorescence emission. During denaturation, the extended probe amplicon 313 is separated from its complement 315. When returned to the single stranded configuration, the label 316 and quencher 318 are brought back into proximity and fluorescence is again quenched.

FIG. 3B is a graph showing the fluorescence signals from the TaqMan probes 302 and the EF probes 312 over time during thermal cycling of an amplification process. The temperatures of the thermal cycling may be varied according to particular application needs. As an example, the denaturation stage may be carried out at a temperature in a range of from about 80° C. to about 100° C., for example from about 90° C. to about 95° C., and the annealing/extension stage may be carried out at a lower temperature, such as in a range from about 40° C. to about 75° C., for example from about 50° C. to about 70° C., for example from about 55° C. to about 65° C. In some implementations, the first set of reaction conditions (e.g., first set of conditions 218, as discussed with reference to FIG. 2A) corresponds to a denaturation stage 318, while the second set of reaction conditions (e.g., second set of conditions 220, as discussed with reference to FIG. 2A) corresponds to an annealing/extension stage 320.

While various embodiments cycle between a denaturation stage 318 and a combined annealing/extension stage 320 (i.e., the amplification process cycles between two target temperatures), other embodiments may include separate annealing and extension stages. In such embodiments, the temperature, and possibly other reaction conditions, may be varied between the annealing and the extension stages. For example, the extension stage can be carried out at a higher temperature than the annealing stage temperature. In some embodiments, the amplification process cycles between two differing target temperatures or two differing target temperature ranges for at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% of the cycles of the amplification process.

FIG. 3B shows that the fluorescence signal associated with the TaqMan probe 302 increases during the extension stage 320 and then remains at a similar level through the denaturation stage 318 of the next cycle, whereas the fluorescence signal associated with the EF probe 304 increases during the extension stage 320 but decreases to the baseline signal level associated with the EF probe 304 once the subsequent denaturation stage 318 reaches the target denaturation temperature. Those having ordinary skill in the art would appreciate that the cycles N, N+1, N+2 of FIG. 3B may begin at a different stage, however, in which case the comparison of signal levels noted above may be shifted.

In some embodiments, the first set of reaction conditions (e.g., the denaturation conditions 318) includes a first measurement temperature at which the first label signal is measured, and the second set of reaction conditions (e.g., the annealing/extension conditions 320) includes a second, different measurement temperature at which the first and second label signal is measured. In some embodiments, the first and second measurement temperatures differ by at least about 10° C. or more, about 15° C. or more, about 20° C. or more, about 25° C. or more, or about 30° C. or more. The first measurement temperature may be the target denaturation temperature in a range of, for example, about 80° C. to about 100° C., for example, from about 90° C. to about 95° C., and the second measurement temperature may be the target annealing/extension temperature in a range of, for example, from about 40° C. to about 75° C., for example, from about 50° C. to about 70° C., for example from about 55° C. to about 65° C.

### Example Techniques and Implementations

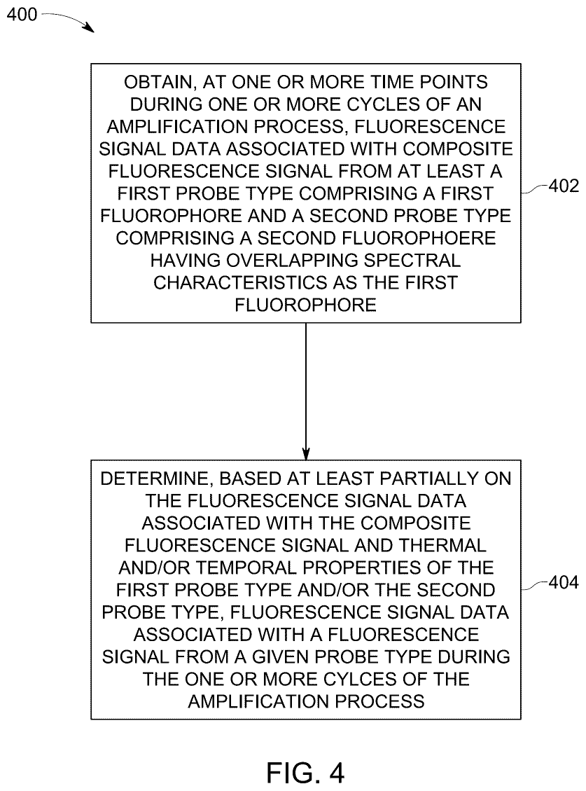

Disclosed techniques for enabling analyte detection in multiplexed amplification processes may entail the performance of various acts. For instance, FIG. 4 illustrates an example flow diagram 400 depicting acts associated with enabling analyte detection in a multiplexed amplification process (e.g., a multiplexed polymerase chain reaction (PCR) process).

Act 402 of flow diagram 400 includes obtaining, at one or more time points during one or more cycles of an amplification process, fluorescence signal data associated with a composite fluorescence signal from at least a first probe type comprising a first fluorophore and a second probe type comprising a second fluorophore which has substantially overlapping spectral characteristics as the first fluorophore. The first probe type and the second probe type may be selected/configured such that their associated label signals respond differently to different sets of reaction conditions (e.g., similar to the first probe 202 and second probe 204 discussed hereinabove with reference to FIG. 2A). For instance, the first probe type and the second probe type may differ in thermal and/or temporal properties. As noted above, the second fluorophore and the first fluorophore may have substantially overlapping spectral characteristics, such that both fluorophores may be configured to generate fluorescence with a substantially similar range of wavelengths (e.g., corresponding to the first label and the second label 212 discussed hereinabove with reference to FIG. 2A).

A composite signal may comprise a fluorescence signal that both the first probe type and second probe type substantially contribute to. As noted above, fluorescence signal data associated with such a composite fluorescence signal (and captured during particular reaction conditions, such as particular temperature conditions) may be regarded as “composite fluorescence signal data”or “second fluorescence signal data”.

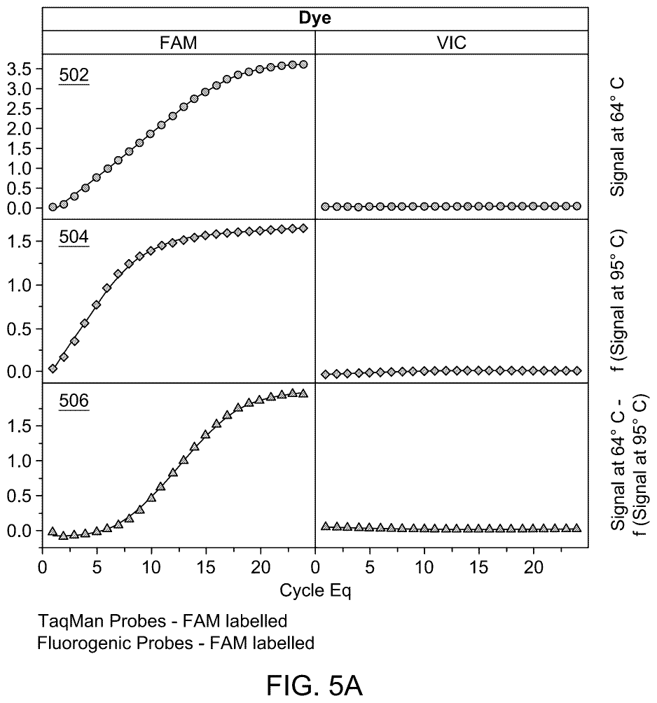

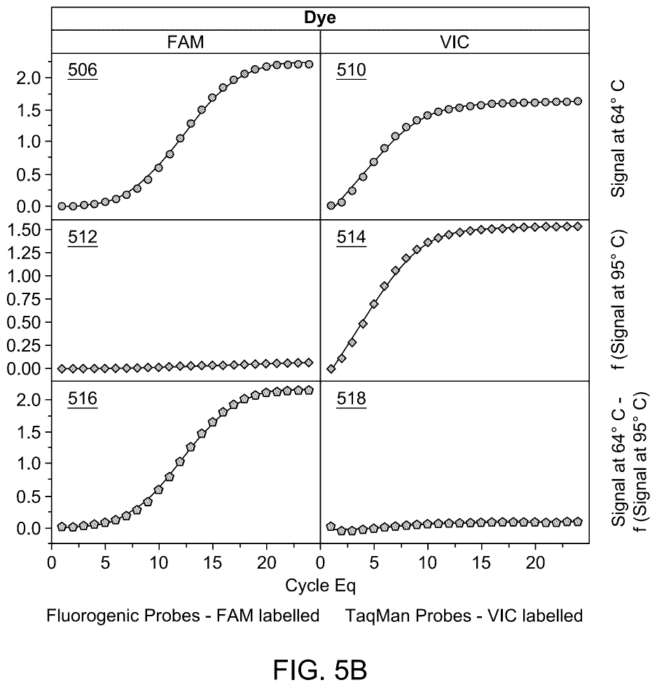

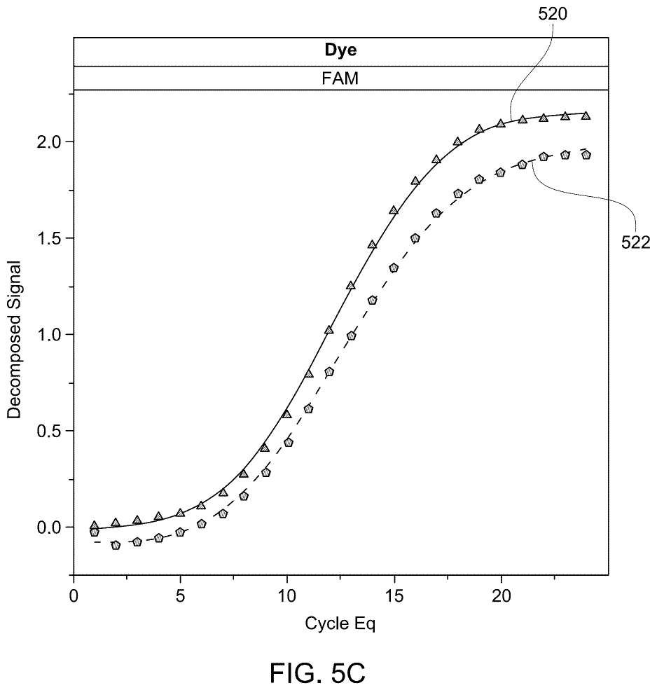

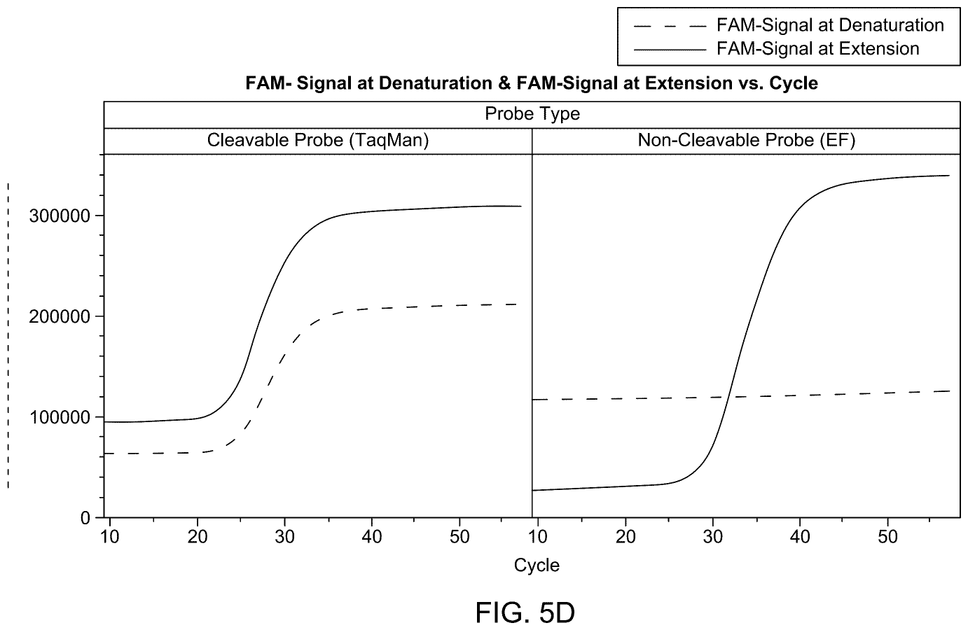

As will be discussed in more detail hereinafter, FIG. 5A provides an example graphical depiction of composite fluorescence signal data from a first probe type and a second probe type (e.g., where TaqMan and EF probes are labeled with FAM fluorophore, the FAM signal at 64° C. (top row) representing a composite fluorescence signal). In some instances, the first fluorophore of the first probe type and the second fluorophore of the second probe type are the same (e.g., in FIG. 5A where both the TaqMan and EF probes are FAM labelled). Notwithstanding the potential commonality between the fluorophores of the two probe types, the two probe types can be otherwise configured to have respective binding affinity to different analytes (e.g., different target nucleic acids). For example, the first probe type may have binding affinity to a first analyte (e.g., corresponding to the first probe 202 being designed to interact with the first target 206, as discussed above with reference to FIG. 2A) and the second probe type may have binding affinity to a second analyte that is different from the first analyte (e.g., corresponding to the second probe 204 being designed to interact with the second target 208, as discussed above with reference to FIG. 2A).

In some implementations, the one probe type (e.g., the first probe type) is a cleavable probe and the other probe type(s) (e.g., the second probe type) is/are non-cleavable. In some implementations, the cleavable probe type is a TaqMan probe, and the non-cleavable probe type is an EF probe (e.g., in the examples discussed with reference to FIGS. 3A, 3B, and 5A through 5D). The first probe type may be associated with a cumulative fluorescence that stays substantially stable across multiple segments of an amplification cycle and increases cumulatively over multiple amplification cycles, whereas the second probe type may be associated with a transient fluorescence that fluctuates significantly over each amplification cycle, such as during differing stages of an amplification cycle.

Act 404 of flow diagram 400 includes determining, based at least partially on the fluorescence signal data associated with the composite fluorescence signal and thermal and/or temporal properties of the first probe type and/or the second probe type, fluorescence signal data associated with a fluorescence signal from a given probe type during the one or more cycles of the amplification process. The given probe type with which the determined fluorescence signal is associated may comprise at least one of the first probe type or the second probe type (e.g., the derived EF-associated signal of FIG. 5C).

In some implementations, first fluorescence signal data is acquired at multiple timepoints during the amplification process from which the composite fluorescence signal data is obtained. The first fluorescence signal data may be associated with different reaction conditions than reaction conditions associated with the composite fluorescence signal data. For instance, the composite fluorescence signal data be associated with a second set of reaction conditions (e.g., corresponding to the second set of conditions 220, 320 discussed above with reference to FIGS. 2A, 2B, and 3B) and the first fluorescence signal data may be associated with a first set of reaction conditions (e.g., corresponding to the first set of conditions 218, 318 discussed above with reference to FIGS. 2A, 2B, and 3B).

In some instances, the different sets of reaction conditions are associated with different temperature conditions, such as different temperatures or different ranges of temperatures (e.g., a first temperature condition associated with the first fluorescence signal data, and a second temperature condition associated with the second or composite fluorescence signal data). In some instances, the first temperature condition is higher than and/or does not overlap with the second temperature condition. For example, FIG. 3B depicts a second temperature condition of 65° C., which may be associated with the composite fluorescence signal data, and a first temperature condition of 95° C., which may be associated with the second fluorescence signal data. Other temperature conditions are within the scope of the present disclosure, (e.g., a second temperature condition within a range of about 40°-75° C.; a first temperature condition within a range of about 80°-100° C.). The first and second temperature conditions can be associated with different stages of the amplification process. For example, the second temperature condition may be associated with an annealing stage or extension stage of the amplification process or a combined extension/annealing stage (e.g., second set of conditions 320 of the various amplification cycles represented in FIG. 3B), whereas the first temperature condition may be associated with a denaturing stage of the amplification process (e.g., first set of conditions 318 of the various amplification cycles represented in FIG. 3B).

In view of the reaction difference in reaction conditions, the composite and first fluorescence signal data may accordingly be associated with different timepoints or time periods within an amplification process. For example, datapoints (or subsets of datapoints) that form the composite fluorescence signal and the first fluorescence signal data may be acquired/captured in a temporally interleaved manner (e.g., alternating between capturing datapoints for the composite fluorescence signal data and the first fluorescence signal data as the reaction conditions alternate during the amplification process).

Determining the fluorescence signal data associated with the fluorescence signal for the given probe type may utilize as inputs the composite fluorescence signal data (discussed above with reference to act 402) and the first fluorescence signal data, as will be described in more detail hereinbelow with reference to FIG. 5A (e.g., referring briefly to FIG. 5A, the signal data in the top row, left column represents example measured composite fluorescence signal data; the signal data in the middle row, left column represents example transformed first fluorescence signal data determined using measured first fluorescence signal data; and the signal data in the bottom row, left column represents example fluorescence signal data associated with the fluorescence signal for the given probe). The fluorescence signal data associated with the fluorescence signal for the given probe may be determined in real-time during the amplification process, or as a post-processing operation.

FIGS. 5A and 5B illustrate results of a qPCR duplex assay in which TaqMan and EF probes were designed to generate fluorescence signals in the same dye channel (FIG. 5A) or in different dye channels (FIG. 5B). In the assay shown in FIG. 5A, both the TaqMan probes and the EF probes were labelled with FAM, and the VIC fluorophore signal thus served as a control. In the assay shown in FIG. 5B, the TaqMan probes were labeled with VIC and the EFs were labelled with FAM. The reaction mixture composition, template DNA concentrations, and amplification conditions were otherwise held the same in the two assays.

In FIG. 5A, graph 502 shows the FAM fluorescence signal over cycle number measured at a temperature during the annealing/extension stage (64° C. in this example). This signal is expected to include fluorescence generated by both the TaqMan probe labels (those that have been cleaved from the probes) and the RF probe labels (those that have been incorporated into double stranded amplicons). The FAM signal of graph 502 of FIG. 5A may comprise composite fluorescence signal data, as discussed above with reference to FIG. 4.

Graph 504 of FIG. 5A shows the FAM fluorescence signal over cycle number measured at a temperature during the denaturation stage (95° C. in this example) and modified by a linear function that correlates the 95° C. measurement to a 64° C. measurement for the TaqMan probes. This signal indicates an approximate fluorescence associated with the TaqMan probes at 95° C. and is expected to include fluorescence generated by the TaqMan probe labels but not to include significant fluorescence from the EF probe labels. The FAM fluorescence signal data measured at 95° C. and linearly transformed to provide the signal data shown in graph 504 of FIG. 5A may comprise “first fluorescence signal data” as discussed as above with reference to FIG. 4. The transformed fluorescence signal data depicted in graph 504 of FIG. 5A may thus be regarded as “transformed first fluorescence data.” As noted above, the function used to correlate the 95° C. measurement to the 64° C. measurement may be determined by comparing TaqMan probe signals measured according to the different temperature conditions (e.g., in the absence of EF or other probe types in the same spectral channel).

In the example of FIG. 5A, the composite fluorescence signal data (e.g., in graph 502 of FIG. 5A) is measured according to a second set of reaction conditions (i.e., at an annealing or extension stage temperature (or combined annealing/extension stages of 64° C.) and captures fluorescence signals from both probe types (i.e., TaqMan and RF probes). The transformed first fluorescence signal data (e.g., of graph 504 of FIG. 5A) approximates the fluorescence signal from one of the probe types (i.e., TaqMan probe type) according to the first set of reaction conditions (i.e., at the denaturation stage temperature of 95° C.). As noted above, the composite fluorescence signal data and the transformed first fluorescence signal data may be used to determine the fluorescence signal from the other probe type (EF probe type) according to the second set of reaction conditions (i.e., at the annealing or extension temperature of 64° C.). The composite fluorescence signal data may be modified by the transformed first fluorescence signal data (e.g., by subtracting the transformed first fluorescence signal data from the composite fluorescence signal data) to generate the fluorescence signal data for the EF probe type (e.g., the “given probe type”as discussed herein).

Graph 506 of FIG. 5A shows the resolved fluorescence signal determined by subtracting the signal of graph 504 (i.e., the transformed first fluorescence signal) from the signal of graph 502 (i.e., the composite fluorescence signal). This signal is expected to estimate the fluorescence generated by the EF labels, separate from fluorescence attributable to the TaqMan probe labels. This signal may thus be used to quantify/analyze a target associated with the EFs in the reaction mixture (e.g., one or more target nucleic acids). The first fluorescence data or the transformed fluorescence signal data may be used to quantify/analyze a different target associated with the TaqMan probes in the reaction mixture.

Accordingly, the disclosed techniques may enable multiple targets/analytes to be advantageously assayed within the same reaction mixture through the same amplification process using probes with fluorophores within the same detection channel (e.g., within the same fluorescence channel), thereby beneficially enabling increases in the plexy of multiplex assays without relying on additional dyes.

The graphs of FIG. 5B represent the same signal measurement types as in FIG. 5A. Graph 508 shows the fluorescence signal generated by the EF labels (FAM), and graph 510 shows the fluorescence signal generated by the TaqMan probe labels (VIC). Graph 512 shows the insignificant change of fluorescence generated by the EF labels at the denaturation temperature (i.e., the EF label fluorescence remains substantially at its baseline signal level), whereas graph 514 shows the fluorescent signal generated by the TaqMan probe labels, which is substantially equal (after application of the linear function) to the signal measured at the annealing/extension temperature. Because the TaqMan and EF probes were differentially labelled in this assay, graph 516 shows a resolved signal for the EF label that essentially matches the EF signal at the annealing/extension temperature (in graph 508), and graph 518 shows the expected zero signal for the TaqMan probe resulting from subtraction of the TaqMan label signal during denaturation from the TaqMan label signal during annealing/extension. The EF-associated fluorescence signal of graph 516 therefore closely represented a direct measurement of the signal from the EF labels (e.g., of graph 508).

FIG. 5C compares the derived EF-associated fluorescence signal 520 (graph 506 of FIG. 5A) as resolved from the assay of FIG. 5A with the measured EF-associated fluorescence signal 522 (graph 516 of FIG. 5B) which represents a direct measurement of EF label fluorescence. The results showed close correlation between the resolved and measured signals. The results therefore showed that fluorescent signals attributable to different probe types using the same label within the same detection channel can be separately resolved.

For further reference, FIG. 5D illustrates the fluorescence signal over cycle number measured during an annealing/extension stage (at a 65° C. temperature condition in this example) and during a denaturation stage (at a 95° C. temperature condition in this example) with TaqMan probe and EF probe compositions.

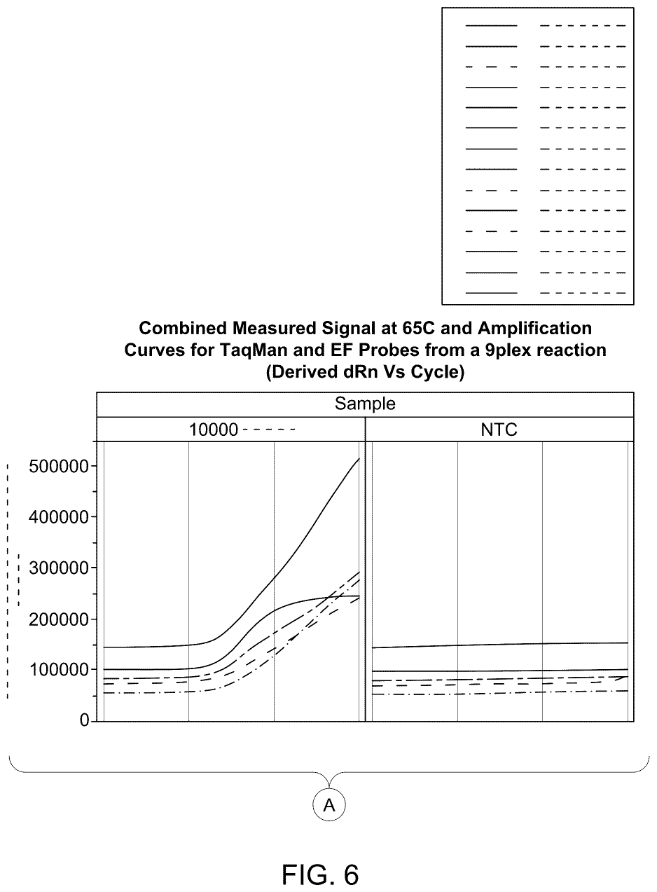

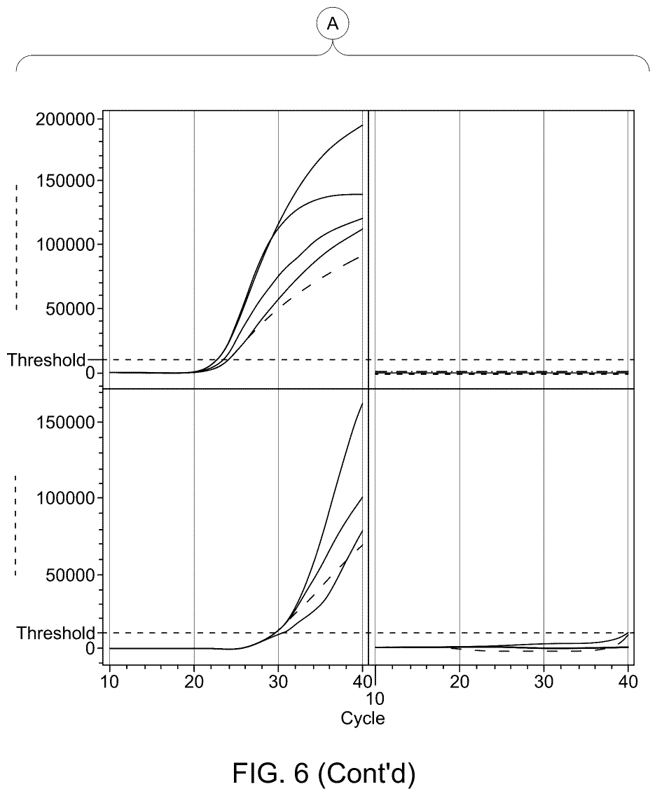

FIG. 6 illustrates the results of another assay test that included 5 different detection channels/dyes, four of which had a TaqMan probe and an EF probe with the same label (dye) in each channel and differing between the four channels. One of the channels had only the TaqMan probe. The results show that the fluorescent signals of the different probe types can be independently determined, and thus that a 9-plex reaction can be effectively carried out.

The workflow of FIG. 4 can also be implemented in a digital PCR (dPCR) multiplex assay by obtaining the signal acquisition at multiple time points of a predetermined endpoint cycle (or other designated cycle at which the PCR process is assumed to be completed and/or a designated cycle at which a signal threshold that is sufficiently above background signal occurs).

In various embodiments, an endpoint cycle in accordance with the present disclosure may range from 20 to 45 cycles, for example, from 30-40 cycles. However, the number of cycles to an endpoint cycle may change and be correlated to where the fluorescence signal indicative of amplification product reaches an approximate plateau.

In the context of a dPCR multiplex assay, for example, for two target analytes and using different probes for detection of the different target analytes, each reaction site into which the sample is segregated can potentially have one of four reaction product populations at the completion of the assay: 1) no amplified reaction product; 2) amplified reaction product corresponding to one of the two target analytes; 3) amplified reaction product corresponding to the other of the two target analytes; and 4) amplified reaction product corresponding to both of the two target analytes. Those having ordinary skill in the art would appreciate however for that sufficiently low concentrations of one of the target analytes, there may be no reaction sites having the fourth population noted above. If two different probe types are used with the same dye or dyes have substantially overlapping spectral emissions, then dPCR will result in four populations of reactions sites: 1) reaction sites that are negative (background signal) associated with no amplified reaction product; 2) reactions sites that emit a signal only at a first set of reaction conditions during the cycle due to the presence of a probe type that emits a signal only under the first set of reaction conditions); 3) reaction sites that emit signal at both a first and a second set of differing reaction conditions during the cycle due to the presence for which another probe type that emits signal under both sets of conditions; and 4) reaction sites that emit signal at both the first and second set of differing reaction conditions during the cycle due to the presence of both probe types which emits.

Accordingly, when utilizing probes having labels that provide overlapping spectra (including probes having the same labels), signal obtained only at one time period during an endpoint cycle of dPCR would not be resolvable to know which reaction sites have only a single target and which target they contained. Similarly, for reaction sites emitting a greater signal level signifying that the reaction site contains multiple amplification products, the overlapping or same emission spectra does not necessarily permit definitive discernment that the amplified product corresponds to there having been two of the same target nucleic acid at the site or two different target nucleic acids at the site.

By exploiting the temporal and/or thermal properties of differing probe types, however, the signal obtained from reaction sites of a dPCR multiplex assay can be resolved to allow for determining: 1) reaction sites containing no reaction product (background signal); 2) reaction sites containing amplified reaction product corresponding to one of the two target analytes; and 3) reaction sites containing amplified reaction product corresponding to the other of the two target analytes. While there may be reaction sites that contain amplified reaction product corresponding to both target analytes, the disclosed techniques for obtaining and analyzing the signal data allows for assigning the respective signals from respective probes to such reaction sites, which thereby can permit concentrations of each to be determined from the starting sample. Moreover, with further data analysis, it permits the ability to render visualizations of the results in a one-dimensional (1-D) fashion to provide a more robust understanding of the data results to a user.

More specifically, as set forth in FIG. 4, and considering the use of a first cleavable probe type and a second non-cleavable probe type as described above with reference to FIGS. 3A and 3B, in one example as applied to dPCR signal acquisition from the multiple reaction sites of a dPCR assay can occur at two stages during the same dPCR cycle, such as an annealing/extension stage (and the associated temperature conditions described above) and the denaturation stage (and the associated temperature conditions described above) within the same PCR cycle, such as an endpoint PCR cycle. Thus, a composite signal can be obtained, as set forth at 402 of FIG. 4, by obtaining signal at the end of the dPCR assay, for example at an endpoint cycle or after a last amplification cycle, for example, where a double stranded amplification product exists. This can correspond in some embodiments to an annealing/extension stage of the endpoint cycle or after and at a temperature where additional denaturation has not occurred. This sense of “composite fluorescence signal” connotes that reaction sites corresponding to signal emission include reaction sites with only the amplified product of first target analyte (and thus first probe), reaction sites with only the amplified product of the second target analyte (and thus second probe), and in some cases reactions sites with the amplified product both the first and second target analytes (and thus the first and second probes). As noted above, fluorescence signal data associated with such a composite fluorescence signal (and captured during particular reaction conditions, such as particular temperature conditions) may be regarded as “composite fluorescence signal data” or “second fluorescence signal data”.

Further, as set forth at 404 of flow diagram 400 of FIG. 4, a determination can be made, based at least partially on the fluorescence signal data associated with the composite fluorescence signal and thermal and/or temporal properties of the first probe type and/or the second probe type, fluorescence signal data at a reaction site associated with a fluorescence signal from a given probe type at the endpoint cycle of the dPCR assay. The given probe type with which the determined fluorescence signal is associated can comprise at least one of the first probe type or the second probe type.

In some implementations, first fluorescence signal data is acquired at one or more timepoints during the same cycle of the dPCR assay (e.g., the endpoint cycle) from which the composite fluorescence signal data is obtained. The first fluorescence signal data may be associated with different reaction conditions during the cycle than the reaction conditions associated with the composite fluorescence signal data. For instance, the composite fluorescence signal data be associated with a second set of reaction conditions (e.g., corresponding to the second set of conditions discussed above with reference to FIG. 2A) and the first fluorescence signal data may be associated with a first set of reaction conditions (e.g., corresponding to the first set of conditions discussed above with reference to FIG. 2A). As noted above, the first set of conditions can correspond to the denaturation stage (and the associated temperature conditions described above) of the PCR cycle while the second set of conditions can correspond to annealing/extension stage (and the associated temperature conditions described above). The first fluorescence signal data corresponds to amplified reaction product of the target nucleic acid associated with the first probe type, e.g., a cleavable probe type, and will not include fluorescence signal data corresponding to amplified reaction product of the target analyte associated with the second probe type, e.g., the non-cleavable probe type. This first fluorescence signal data can thus allow for determining reaction sites containing the amplified product of one of the target nucleic acids, which includes reaction sites that contain only the amplified product of the target analyte associated with the first probe type and reaction sites that contain the amplified product of the both the target analytes, associated with both the first and second probe types.

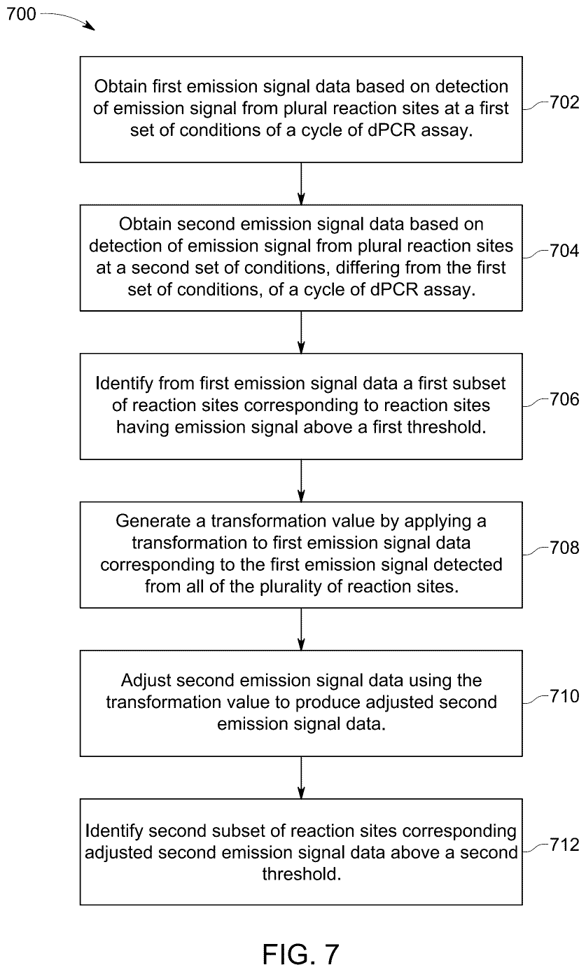

With reference to FIG. 7, a flow diagram for an embodiment of a dPCR analysis workflow to enable assignment of the signal to the different amplified products of different target analytes using the same detection channel (e.g., the same label or label with overlapping emission spectra) is depicted. At action 702, first emission signal data is obtained based on detection of emission signal from a plurality of reaction sites of a dPCR assay at a first set of conditions of a cycle of the dPCR assay. For example, fluorescence signal data may be obtained from fluorescence emission from the reaction sites at a first stage, such as a denaturation stage, of an endpoint dPCR cycle. At such a stage, and when using a cleavable probe designed to bind with one of the target analytes in the sample, the signal associated with the reaction sites will include background signal from some of the sites with no amplified product and sites with amplified product of the target analyte interacting non-cleavable probe type, and the signal associated with elevated signal from some of the sites with amplified product of a target analyte interacting with a cleavable probe type.

At action 704, second emission signal data is obtained based on detection of emission signal from a plurality of reaction sites of a dPCR assay at a second set of conditions, differing from the first set of conditions, of the cycle (e.g., the same cycle as in 702) of the dPCR assay. For example, fluorescence signal data may be obtained from fluorescence emission from the reaction sites at a second stage, such as an annealing or extension stage (or a combined annealing/extension stage), of an endpoint dPCR cycle, or at any temperature after such cycle where double-stranded amplification product exists. At such a stage, and when using a cleavable probe designed to bind with one of the target analytes in the sample and a non-cleavable probe designed to bind with another of the target analytes, the signal associated with the reaction sites will include background signal from some of the sites with no amplified product, and the signal associated with elevated signal from reaction sites with amplified product of the target analyte interacting with a cleavable probe type and reaction sites with amplified product of the other target analyte interacting with the non-cleavable probe type, of which reaction sites, some may have one or the other of the target analytes and some may have both.

Due to either the same or substantially overlapping spectra, it can be difficult to discern which of the sites have which target(s) from such data alone. Thus, workflow 700 further can include at action 706 identifying from the first emission signal data, a first subset of reaction sites corresponding to reaction sites having emission signal above a first threshold. This first threshold can be set based on a signal level of the background signal so that it can be reliably determined that signal above the threshold is indicative of reaction sites that contain amplified product of the target analyte associated with the probe type that responds to the first set of reaction conditions, such as for example, the target analyte that interacts with the cleavable probe type.

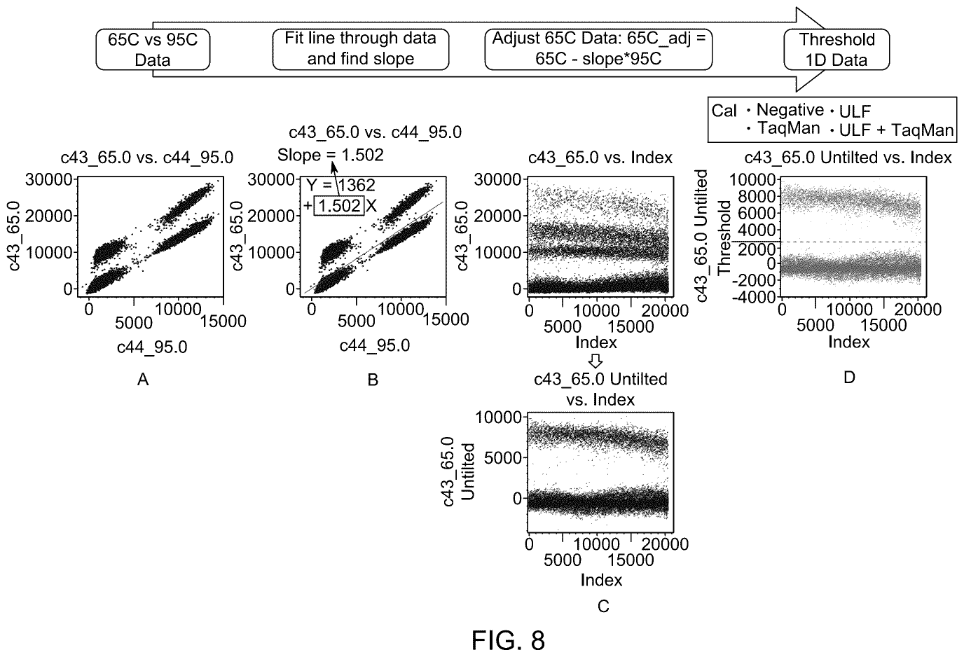

At action 708, generate a transformation value by applying a transformation to the first emission signal data corresponding to the first emission signal detected from all of the plurality of reaction sites. This can allow for a derived signal to be obtained which estimates the equivalent emission signal from these same reaction sites that are also detected as part of the second emission signal data obtained under the second set of conditions. In other words, because the emission signal from the negative (background signal) reaction sites and the first subset of reaction sites will also be present when detecting the second emission signal, the transformation can be used to account for that. In various embodiments, the transformation can be considered a correction factor to be applied to the first emission signal data, which may be obtained, for example, by fitting a line through the data and determining the slope, as further illustrated and described below with reference to FIG. 8. And thus the transformation value can be the correction factor multiplied by the first fluorescence emission signal data.

At action 710, the second emission signal data can be adjusted based on the transformation value to produce adjusted second emission signal data. For example, the second emission signal data can be adjusted by subtracting the transformed first emission signal data (e.g., the transformation value). At action 712, a second subset of the plurality of reaction sites can be identified from the adjusted second emission signal data that have adjusted second emission signal above a second threshold. This second subset of plurality of reaction sites corresponds to the reaction sites containing the second probe type interacting with the other target analyte. For example, the reaction sites of the dPCR assay that contain a target analyte associated with the non-cleavable probe type, which can include reaction sites that also contain the other target analyte associated with the cleavable probe type. In some case, for example, such as when this one target analyte has a sufficiently low concentration, particularly in comparison to the other target analyte, it is expected that the low concentration target analyte will generally be at a reaction site that also includes the higher concentration target analyte.

The dPCR technique described above can be utilized to provide robust data visualization of a multiple dPCR assay which obtains emission signals from multiple analytes within one detection channel. More specifically, various implementations can allow for a 1-D visualization of the data for concentration determination, thereby providing for robust thresholding of positive and negative populations of different targets at different reaction conditions. FIG. 8 is representative of data visualizations that can be output for dPCR assays using techniques such as that described above and with reference to FIG. 7, and which permits a data visualization in two-dimensions (2-D) corresponding to first fluorescence signal data on one axis (e.g., the X-axis) and second fluorescence signal data on an orthogonal axis (e.g., the Y-axis).

In plot A of FIG. 8, fluorescence emission signal data from a plurality of a reaction sites of an endpoint cycle of a dPCR reaction obtained at two different temperatures (65° C. and 95° C.) is plotted. The signal data corresponds to detection of fluorescence from a single detection channel. From the data plotted, four populations of reaction sites can be seen by the four clusters of data points. Beginning with the cluster at the bottom left, the cluster corresponds to reaction sites having no amplified reaction product (background signal). The cluster at the top left corresponds to reaction sites having only amplified product of a target analyte that interacts with a labeled non-cleavable probe (e.g., an extendable hairpin probe (EF)). The cluster at the bottom right portion corresponds to reaction sites having amplified reaction product of another, different target analyte, that interacts with a labeled cleavable probe (e.g., a TaqMan probe). The cluster at the top right portion correspond to reaction sites having amplified reaction product of both the target analytes. While the data visualization in A of FIG. 8 provides four fairly discernible clusters, many assays will not produce such results and the data points may be much more blurred, making such discernment impractical at best.

From the data in A, however, a transformation can be derived from the data points, such as by fitting a line through the data as shown in B of FIG. 8. Due to the differing axes, the slope of the fit line can provide a correction factor to adjust the second emission signal data (i.e., corresponding to fluorescence emission at 65° C. in the example of FIG. 8), which is based on emission occurring from reaction sites that contain either or both of the amplified product of the different target analytes that interact with the cleavable and non-cleavable probes. The plots C of FIG. 8 show the correction factor applied to all of the first fluorescence emission signal (i.e., the slope determined at B multiplying all the data points corresponding to the 95° C. data) and the corrected data points replotted (top plot). In other words, the top plot of C contains the 65° C. data points from B and the corrected 95° C. data points. The bottom plot of C of FIG. 8 then shows adjusted data points of the top plot of C by subtracting from those points a value equal to the correction factor times the first emission signal data (i.e., the signal data at 95° C.). As can be seen from FIG. 8, the plots of C are thus transformed to be visualized in 1-D, with clearly separated clusters of data.

With the transformation to the 1-D data, thresholds can be applied to further identify reaction sites containing no amplified product of the target analyte interacting with the non-cleavable probe (EF probe in FIG. 8D). As can be seen by FIG. 8D, the reaction sites containing only amplified product of the target analyte interacting with the cleavable probe (TaqMan in 8D) are grouped with the reaction sites containing no amplification produce (lower cluster of data points in FIG. 8D). The reaction sites above the threshold (identified by the upper cluster of data points in FIG. 8D) thus correspond to reaction sites containing other target analyte interacting with the non-cleavable probe (EF probe); in other words, the reaction sites containing amplified product of only the target interacting with the EF probe or containing amplified product of both targets.

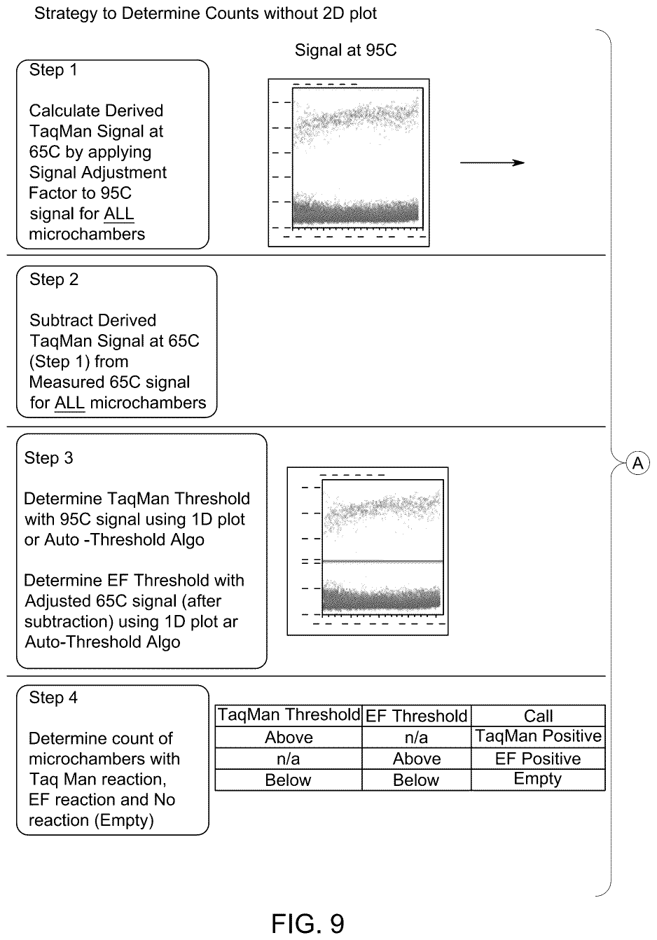

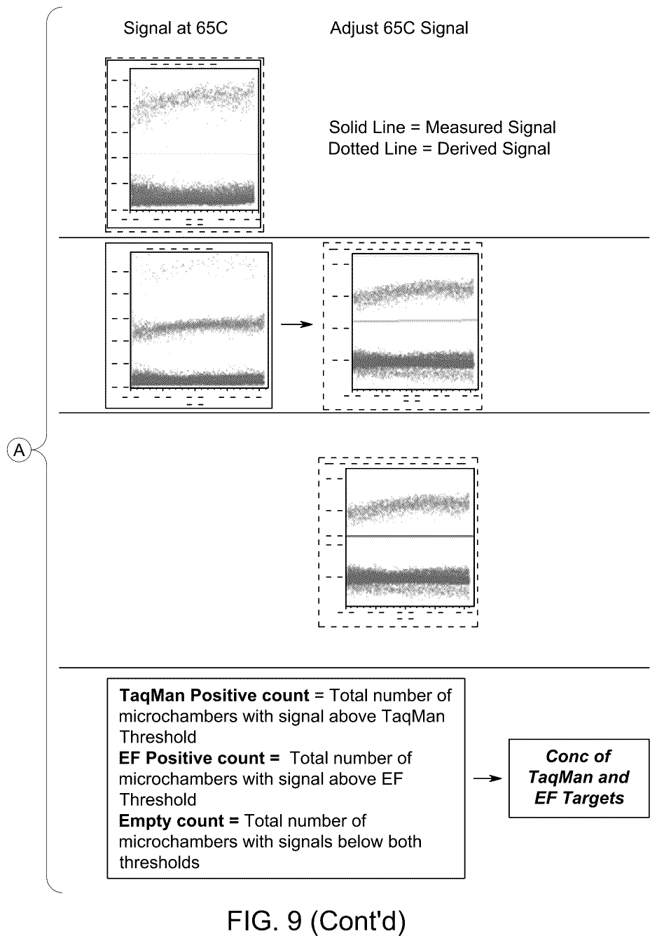

FIG. 9 provides an example implementation for dPCR data analysis and visualization that can determine calls of reaction sites (quantification/concentration of differing target analytes) without the 2-D plot of A of FIG. 8. In FIG. 9, the emission signal data is obtained using the techniques described above and as for the data of FIG. 8. In Step 1 of FIG. 9, first fluorescence signal data from all the reaction sites of the dPCR assay taken at a denaturation stage of an endpoint cycle (i.e., at 95° C.) is plotted (represented as the measured solid line highlighted plot at Step 1). The derived corresponding emission signal for all reaction sites at 65° C. is then determined by applying the correction factor to the measured data and plotted as the derived signal data at Step 1 of FIG. 9 (represented as the dotted line highlighted data). The plots of FIG. 9 depict the reference location (index) of the reaction sites in the array on the X-axis versus the fluorescence signal on the Y-axis.

At Step 2, the derived signal data for all the reactions sites from the derived signal plot of Step 1 is then subtracted from the second fluorescence emission signal data obtained from all of the plurality of reaction sites of the dPCR assay (the latter being reflected by the solid line highlighted (measured) signal data plot of Step 2). This leads to the data set plotted in dotted line highlighted (derived) plot of Step 2 of FIG. 9.

At Step 3, a first threshold can be determined based on the data plot of the first fluorescence emission signal data (the data collected during the denaturation stage at 95° C. in the data sets shown). This again is represented by the solid line measured data plot at Step 3, which is the same as the solid line measured data plot of Step 1, except with the Step 3 data indicating the determined threshold signal value by the solid line across the plot. Similarly, a second threshold can be determined using the dotted line derived signal data of Step 2, shown again in Step 3 except with the determined second threshold signal value represented by the solid line across the plotted data. The first and second threshold signal values can be selected by a user based on choosing a well-defined region that separates the two data clusters of the plot or by an algorithm or other modeling scheme.

At Step 4 of FIG. 9 a count of each of the reaction sites containing no amplified product, the reaction sites containing target analyte interacting with the cleavable probe, and the reaction sites containing a different target analyte interacting with the non-cleavable probe can be determined by applying the first and second threshold values to all of the data. From this information, concentration of the first and second target analyte in the sample subjected to the multiplex dPCR assay can be determined. More specifically, for the example of FIG. 9, the reaction sites counted as containing the target analyte interacting with the TaqMan probe are the reaction sites having a fluorescence signal above the first threshold. The reaction sites counted as containing the other target analyte interacting with the EF probe are the reaction sites having a fluorescence signal above the second threshold.

For the various dPCR implementations described, the transformation and transformation values (e.g., linear transformation and slopes) can be predetermined by a separately run dPCR single plex or multiplex assay that uses a sample with only the target analyte that interacts with the probe type that presents the label signal at the higher temperature (e.g., the cleavable probe type). Alternatively, it can be determined based on real-time data collected during a qPCR or dPCR assay.

In view of the foregoing, in some implementations, disclosed embodiments may include obtaining different fluorescence signals at different temperatures or time periods of an amplification process (e.g., a PCR process) that involves multiple different probe types. The different probe types may comprise differential fluorescence responses at the different temperatures or time periods. The differential fluorescence responses of the different probe types at the different temperatures or time periods may be used to determine isolated fluorescence signals for the different probe types. This can therefore enable multiplex PCR assays that can quantify different target analytes utilizing emission signals that emit the same or substantially overlapping emission spectra, and thus, for example, utilizing a common detection channel for detecting multiple analytes.

### Extendable Fluorogenic Probe Template Formation

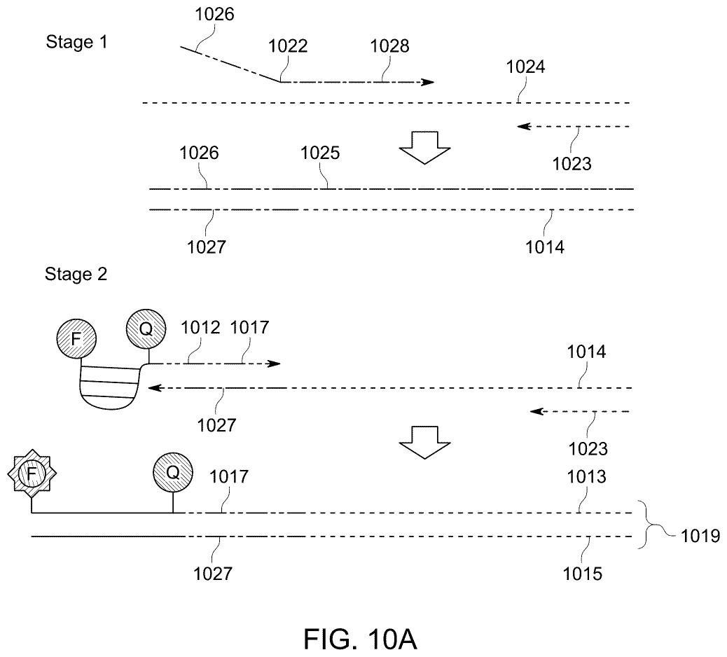

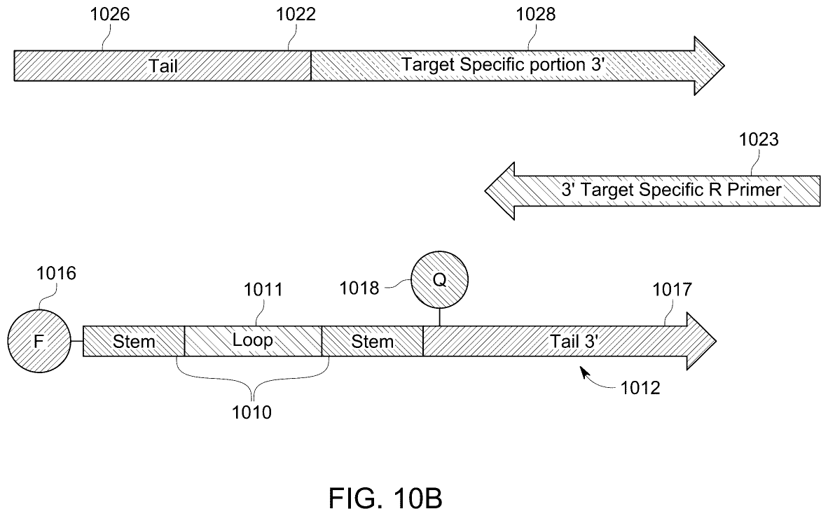

FIG. 10A illustrates a process of using a tailed primer 1022, which is specific to a nucleic acid target 1024, to form the target template 1014 to which the EF probe 1012 can hybridize. The tailed primer 1022 includes a tail 1026 and a target-specific portion 1028. FIG. 10B illustrates an example of the tailed primer 1022 as a forward primer, a target specific primer 1023 paired with the tailed primer 1022 as a reverse primer, and a more detailed view of the EF probe 1012.

As shown, in a first stage, the target-specific portion 1028 hybridizes to the target 1024. Extension of the target-specific portion 1028 forms a tailed amplicon 1025. Primer 1023, which is paired with the tailed primer 1022, enables extension of the complement of the tailed amplicon 1025. It is this complement that forms the target template 1014. As shown, the target template 1014 includes a tail complement portion 1027.

In a second stage, the EF probe 1012 hybridizes to the target template 1014 and amplification can continue, for example, as shown in FIG. 3A. As shown, the EF probe 1012 includes a probe tail 1017 that has substantial homology with the tail 1026 and is therefore complementary to the tail complement portion 1027 of the target template 1014. Extension of probe 1012 and target template 1014 forms a double stranded amplicon 1019. The primer 1023, shown here paired with the tailed primer 122, may also function as the primer 1023 that pairs with the EF probe 11012 to enable formation of the double stranded amplicon 1019, as shown in FIG. 3A.

As shown in FIG. 10B, the tail 1026 can form the 5′ end of the tailed primer 1022. The EF probe 1012 can include a stem-loop portion, with stem portions 1010 on either side of a loop portion 1011, configured to form a stem-loop structure when the EF probe 1012 is single stranded. For example, the label 1016 may be located on one side of the stem-loop portion and the quencher 1018 may be located on the opposite side of the stem-loop portion such that the label 1016 and quencher 1018 are brought into proximity when the stem-loop structure is formed but spaced farther apart when the EF probe 1012 is constrained into a more linear configuration (e.g., when incorporated into a double stranded amplicon).

In the illustrated embodiment, the label 1016 is located at or near the 5′ end of the EF probe 1012 and the quencher 1018 is located 3′ of the label 1016. The positions of the label 1016 and quencher 1018 may be reversed in other embodiments. Preferably, as shown, the stem-loop portion is disposed 5′ of the probe tail 1017 so that the stem-loop portion remains at the end of the amplicons resulting from extension of the EF probe 1012, so that stem-loop structure formation (when single stranded) is less likely to be compromised.

In addition to or alternative to the EF probes described herein, some embodiments may include other labelled oligonucleotides that generate increased fluorescence upon being incorporated into a double stranded amplicon (relative to when in a single stranded state), such as, for example during extension and/or annealing stages of a PCR process. For example, LUX™ primers include an internal fluorophore that is quenched by a hairpin structure located 5′ of the fluorophore. As with EF probes, a LUX™ primer provides increased fluorescence when incorporated into a double-stranded amplicon and the hairpin structure is linearized. Further, any of the primers or probes described herein may include one or more locked nucleic acids (LNAs) as are known in the art.

In some embodiments, the tailed primer 1022 and the corresponding (non-tailed) primer 1023 are provided at different concentrations. For example, the primer 1023 may be provided at a higher concentration than the tailed primer 1022. For example, the primer 1023 may be provided at a concentration that is about 2× (2 times) to about 30× the concentration of the tailed primer 1022, or about 5X to about 25X the concentration of the tailed primer 1022, or about 10× to about 20× the concentration of the tailed primer 1022. Because the primer 1023 can function to both (1) drive the formation of the target template 1014 (as shown in FIG. 10A) and (2) drive the formation of the complement 1015 of the extended probe amplicon 1013 (as shown in FIG. 3A), providing it at a higher concentration than the corresponding tailed primer 1022 can beneficially balance the reaction and help drive overall reaction efficiency.

In some embodiments, the EF probe 1012 is provided at a concentration that is different from the concentration of the tailed primer 1022 and/or the concentration of the primer 1023. For example, the EF probe 1012 may be provided at a concentration that is greater than the concentration of the tailed primer 1022 and that is less than the concentration of primer 1023. In some embodiments, the EF probe 1012 is provided at a concentration that is about 2× to about 20× the concentration of the tailed primer 1022, or about 3× to about 15× the concentration of the tailed primer 1022. As discussed above, providing the primer 1023 at a relatively higher concentration helps to drive the overall efficiency of the reaction. Providing the EF probe 1012 at a concentration that is higher than the tailed primer 1022, but not necessarily higher than the primer 1023, pushes more of the associated amplification toward the EF probe 1012 as opposed to the tailed primer 1022, yet still allows the primer 1023 to function as the primary driver of reaction efficiency.

In addition to or alternative to the “universal” EF probes that use a probe tail 1017, other embodiments include and/or utilize EF probes with a target-specific portion rather than a probe tail 1017. Such EF probes can directly hybridize to a target template nucleic acid as shown in FIG. 10A for generating a target template 114 with a tail complement portion 1027. In such embodiments, the probe tail 1017 of the EF probe 1012 is replaced with a target-specific portion that directly hybridizes to the target 1024. The process is otherwise similar to that shown in FIG. 3A. That is, after the EF probe is extended, a subsequent round of annealing/extension will extend the complement strand, forming the double stranded amplicon that separates the fluorophore and the quencher to allow for fluorescence signal generation.

### Additional Label/Dye Details

Exemplary nonlimiting detectable labels that may be utilized with the embodiments described herein include, for example:

Fluoresceins (e.g., 5-carboxy-2,7-dichlorofluorescein, 5-Carboxyfluorescein (5-FAM), 6-JOE, 6-carboxyfluorescein (6-FAM), VIC, FITC, 6-carboxy-4′,5′-dichloro-2′,7′-dimethoxy-fluorescein (JOE)), 5 and 6-carboxy-1,4-dichloro-2′,7′-dichloro-fluorescein (TET), 5 and 6-carboxy-1,4-dichloro-2′,4′,5′,7′-tetra-chlorofluorescein, HEX, PET, NED, Oregon Green (e.g. 488, 500, 514));

Pyrenes; (e.g. Cascade Blue; Alexa Fluor 405);

Coumarins; (e.g. Pacific Blue, Atto 425, Alexa Fluor 350, Alexa Fluor 430);

Cyanine Dyes; (e.g. Cy dyes such as Cy3, Cy3.18, Cy3.5, Cy5, Cy5.18, Cy5.5, Cy7);

Rhodamines; (e.g., 110, 123, B, B 200, BB, BG, B extra, 5 and 6-carboxytetramethylrhodamine (5-TAMRA, 6-TAMRA), 5 and 6-Carboxyrhodamine 6G, Lissamine, Lissamine Rhodamine B, Rhod-2, ROX (6-carboxy-X-rhodamine), 5 and 6-ROX (carboxy-X-rhodamine), Sulphorhodamine B can C, Sulphorhodamine G Extra, 5 and 6 TAMRA (carboxytetramethyl-rhodamine), (TRITC), ABY, JUN, LIZ, RAD, RXJ, Texas Red; and Texas Red-X);

Alexa Fluor fluorophores (which is a broad class including many dye types such as cyanines) (e.g., Alexa 350, 405, 430, 488, 500, 514, 532, 546, 555, 568, 594, 610, 633, 635, 647, 660, 676, 680, 700, 750);

FRET donor/acceptor pairs (e.g., fluorescein/fluorescein, fluorescein/rhodamine, fluorescein/cyanine, rhodamine/cyanine, fluorescein/Alexa Fluor, Alexa Fluor/rhodamine); and other types of dyes known to those of skill in the art.

Fluorophore labels may be associated with quenchers such as dark fluorescent quencher (DFQ), black hole quenchers (BHQ), Iowa Black, QSY7, QSY21 quencher, Dabsyl and Dabcel sulfonate/carboxylate quenchers, and MGB-NFQ quenchers. Fluorophore labels may also include sulfonate derivatives of fluorescein dyes with SO3 instead of the carboxylate group, phosphoramidite forms of fluorescein, and/or phosphoramidite forms of Cy5, for example.

### Additional Amplification Details

Amplified products resulting from use of one or more embodiments described herein can be generated, detected, and/or analyzed on any suitable platform. In some embodiments, the nucleic acid targets may be single-stranded, double-stranded, or any other nucleic acid molecule of any size or conformation. The amplification processes described herein can include PCR (see, e.g., U.S. Pat. No. 4,683,202). In some embodiments, the PCR is real time or quantitative PCR (qPCR). In some embodiments, the PCR is an end point PCR. In some embodiments, the PCR is digital PCR (dPCR).

In some embodiments, the amplification process includes RT-PCR. A disclosed method may include, for example, subjecting the target nucleic acid to a reverse transcription reaction prior to amplification via PCR. In some embodiments, the amplification process includes one-step RT-PCR (e.g., in a single vessel or reaction volume) in which one or more reverse transcriptases are used in combination with one or more DNA polymerases.

Optionally, certain qPCR assays can be plated into individual wells of a single array or multi-well plate, such as for example a TaqMan Array Card (see, e.g., Thermo Fisher Scientific, Waltham, MA; Catalog Nos. 4346800 and 4342265) or a MicroAmp multi-well (e.g., 96-well, 384-well) reaction plate (see, e.g., Thermo Fisher Scientific, Waltham, MA; Catalog Nos. 4346906, 4366932, 4306737, 4326659 and N8010560). Optionally, the different qPCR assays present in different wells of an array or plate can be dried or freeze-dried in situ and the array or plate can be stored or shipped prior to use. In some embodiments, the concepts described herein may be utilized in in situ hybridization applications not necessarily associated with PCR.

Other amplification methods, such as, e.g., loop-mediated isothermal amplification (“LAMP”), and other isothermal methods are also contemplated for use with the assay embodiments described herein.

The components described herein for enabling intra-channel multiplexing may be provided in a kit along with one or more additional components to enable an amplification process. Such components can include, for example, dNTPs, DNA polymerase, amplification buffers/reagents, master mix components as known in the art, and other components known in the art for enabling or assisting nucleic acid amplification.

### Additional Implementation Details

The principles disclosed herein may be implemented in various formats. For example, the various techniques discussed herein may be performed as a method that includes various acts for achieving particular results or benefits (e.g., the actions of flow diagram 400 of FIG. 4 or of flow diagram 700 of FIG. 7). One contemplated implementation can combine data collected at multiple timepoints of multiple PCR cycles to provide useful data analysis for qPCR while also providing the data analysis from the multiple timepoints of an end cycle of dPCR in accordance with the embodiments outlined above. In addition, data can be collected at end-point of qPCR assay and analyzed in accordance with the techniques described herein, which may be employed, for example, in genotyping applications. Thus, the techniques and data analysis/visualizations disclosed are not mutually exclusive and can be utilized in combination. In some instances, the techniques discussed herein are represented in computer-executable instructions that may be stored on one or more hardware storage devices. The computer-executable instructions may be executable by one or more processors to carry out (or to configure a system to carry out) the disclosed techniques. In some embodiments, a system may be configured to send the computer-executable instructions to a remote device to configure the remote device for carrying out the disclosed techniques.

Systems for implementing the disclosed embodiments may include various components, such as, by way of non-limiting example, processor(s), storage, sensor(s), I/O system(s), communication system(s), etc.

The processor(s) may comprise one or more sets of electronic circuitries that include any number of logic units, registers, and/or control units to facilitate the execution of computer-readable instructions (e.g., instructions that form a computer program). Such computer-readable instructions may be stored within storage. The storage may comprise physical system memory and may be volatile, non-volatile, or some combination thereof. Furthermore, storage may comprise local storage, remote storage (e.g., accessible via communication system(s) or otherwise), or some combination thereof.

In some implementations, the processor(s) may comprise or be configurable to execute any combination of software and/or hardware components that are operable to facilitate processing using machine learning models or other artificial intelligence-based structures/architectures. For example, processor(s) may comprise and/or utilize hardware components or computer-executable instructions operable to carry out function blocks and/or processing layers configured in the form of, by way of non-limiting example, single-layer neural networks, feed forward neural networks, radial basis function networks, deep feed-forward networks, recurrent neural networks, long-short term memory (LSTM) networks, gated recurrent units, autoencoder neural networks, variational autoencoders, denoising autoencoders, sparse autoencoders, Markov chains, Hopfield neural networks, Boltzmann machine networks, restricted Boltzmann machine networks, deep belief networks, deep convolutional networks (or convolutional neural networks), deconvolutional neural networks, deep convolutional inverse graphics networks, generative adversarial networks, liquid state machines, extreme learning machines, echo state networks, deep residual networks, Kohonen networks, support vector machines, neural Turing machines, and/or others.

In some instances, actions performable by a system may rely at least in part on communication system(s) for receiving information from remote system(s), which may include, for example, separate systems or computing devices, sensors, and/or others. The communications system(s) may comprise any combination of software or hardware components that are operable to facilitate communication between on-system components/devices and/or with off-system components/devices. For example, the communications system(s) may comprise ports, buses, or other physical connection apparatuses for communicating with other devices/components. Additionally, or alternatively, the communications system(s) may comprise systems/components operable to communicate wirelessly with external systems and/or devices through any suitable communication channel(s), such as, by way of non-limiting example, Bluetooth, ultra-wideband, WLAN, infrared communication, and/or others.

A system may comprise or be in communication with sensor(s). Sensor(s) may comprise any device for capturing or measuring data representative of perceivable or detectable phenomena. By way of non-limiting example, the sensor(s) may comprise one or more light sensors/detectors, microphones, thermometers, barometers, magnetometers, accelerometers, gyroscopes, and/or others.

Furthermore, a system may comprise or be in communication with I/O system(s). I/O system(s) may include any type of input or output device such as, by way of non-limiting example, a touch screen, a mouse, a keyboard, a controller, a speaker and/or others, without limitation. For example, the I/O system(s) may include a display system that may comprise any number of display panels, optics, laser scanning display assemblies, and/or other components. One will appreciate, in view of the present disclosure, that the sensor(s) may, in some instances, be utilized as I/O system(s).

Disclosed embodiments may comprise or utilize a special purpose or general-purpose computer including computer hardware, as discussed in greater detail below. Disclosed embodiments also include physical and other computer-readable media for carrying or storing computer-executable instructions and/or data structures. Such computer-readable media can be any available media that can be accessed by a general-purpose or special-purpose computer system. Computer-readable media that store computer-executable instructions in the form of data are one or more “physical computer storage media” or “hardware storage device(s).” Computer-readable media that merely carry computer-executable instructions without storing the computer-executable instructions are “transmission media.” Thus, by way of example and not limitation, the current embodiments can comprise at least two distinctly different kinds of computer-readable media: computer storage media and transmission media.

Computer storage media (aka “hardware storage device”) are computer-readable hardware storage devices, such as RAM, ROM, EEPROM, CD-ROM, solid state drives (“SSD”) that are based on RAM, Flash memory, phase-change memory (“PCM”), or other types of memory, or other optical disk storage, magnetic disk storage or other magnetic storage devices, or any other medium that can be used to store desired program code means in hardware in the form of computer-executable instructions, data, or data structures and that can be accessed by a general-purpose or special-purpose computer.

A “network” may comprise one or more data links that enable the transport of electronic data between computer systems and/or modules and/or other electronic devices. When information is transferred or provided over a network or another communications connection (either hardwired, wireless, or a combination of hardwired or wireless) to a computer, the computer properly views the connection as a transmission medium. Transmission media can include a network and/or data links which can be used to carry program code in the form of computer-executable instructions or data structures and which can be accessed by a general purpose or special purpose computer. Combinations of the above are also included within the scope of computer-readable media.

Further, upon reaching various computer system components, program code means in the form of computer-executable instructions or data structures can be transferred automatically from transmission computer-readable media to physical computer-readable storage media (or vice versa). For example, computer-executable instructions or data structures received over a network or data link can be buffered in RAM within a network interface module (e.g., a “NIC”), and then eventually transferred to computer system RAM and/or to less volatile computer-readable physical storage media at a computer system. Thus, computer-readable physical storage media can be included in computer system components that also (or even primarily) utilize transmission media.

Computer-executable instructions comprise, for example, instructions and data which cause a general-purpose computer, special purpose computer, or special purpose processing device to perform a certain function or group of functions. The computer-executable instructions may be, for example, binaries, intermediate format instructions such as assembly language, or even source code.

Disclosed embodiments may comprise or utilize cloud computing. A cloud model can be composed of various characteristics (e.g., on-demand self-service, broad network access, resource pooling, rapid elasticity, measured service, etc.), service models (e.g., Software as a Service (“SaaS”), Platform as a Service (“PaaS”), Infrastructure as a Service (“IaaS”), and deployment models (e.g., private cloud, community cloud, public cloud, hybrid cloud, etc.).

Those skilled in the art will appreciate that the invention may be practiced in network computing environments with many types of computer system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, pagers, routers, switches, wearable devices, and the like. The invention may also be practiced in distributed system environments where multiple computer systems (e.g., local and remote systems), which are linked through a network (either by hardwired data links, wireless data links, or by a combination of hardwired and wireless data links), perform tasks. In a distributed system environment, program modules may be located in local and/or remote memory storage devices.

Alternatively, or in addition, the functionality described herein can be performed, at least in part, by one or more hardware logic components. For example, and without limitation, illustrative types of hardware logic components that can be used include Field-programmable Gate Arrays (FPGAs), Program-specific Integrated Circuits (ASICs), Application-specific Standard Products (ASSPs), System-on-a-chip systems (SOCs), Complex Programmable Logic Devices (CPLDs), central processing units (CPUs), graphics processing units (GPUs), and/or others.

As used herein, the terms “executable module,” “executable component,” “component,” “module,” or “engine” can refer to hardware processing units or to software objects, routines, or methods that may be executed on one or more computer systems. The different components, modules, engines, and services described herein may be implemented as objects or processors that execute on one or more computer systems (e.g., as separate threads).

One will also appreciate how any feature or operation disclosed herein may be combined with any one or combination of the other features and operations disclosed herein. Additionally, the content or feature in any one of the figures may be combined or used in connection with any content or feature used in any of the other figures. In this regard, the content disclosed in any one figure is not mutually exclusive and instead may be combinable with the content from any of the other figures.

The described embodiments are to be considered in all respects only as illustrative and not restrictive. All changes which come within the meaning and range of equivalency of the claims are to be embraced within their scope.

1-112. (canceled)

113. A method for enabling analyte detection in a multiplexed polymerase chain reaction (PCR) assay, the method comprising:
receiving first emission signal data corresponding to emission signal detected from a plurality of reaction sites containing discrete volumes of a sample subjected to a PCR assay, the first emission signal data corresponding to emission signal detected from the plurality of reaction sites at a first stage of a cycle of the PCR assay;
receiving second emission signal data corresponding to emission signal detected from the plurality of reaction sites containing discrete volumes the sample subjected to the PCR assay, the second emission signal data corresponding to emission signal detected from the plurality of reaction sites at a second stage of the cycle of the PCR assay, the second stage differing from the first stage;
identifying, from the first emission signal data, a first subset of reaction sites corresponding to reaction sites having emission signal above a first predetermined threshold;
generating a transformation value by applying a transformation to the first emission signal data of the plurality of reaction sites;
generating adjusted second emission signal data using the transformation value; and
identifying, from the adjusted second emission signal data, a second subset of reaction sites corresponding to reaction sites having emission signal above a second predetermined threshold.

114. The method of claim 113, wherein the first and second emission signal data correspond to emission signal from a first fluorophore and a second fluorophore having overlapping spectral emission characteristics.

115. The method of claim 114, wherein the first and second fluorophore are the same.

116. The method of claim 114, wherein the multiplexed PCR assay comprises a first target analyte and a second target analyte, wherein the first target analyte reacts with a first type of probe carrying the first fluorophore and the second target analyte reacts with a second type of probe carrying the second fluorophore, wherein the first type of probe and the second type of probe differ from each other.

117. The method of claim 116, wherein the first type of probe differs from the second type of probe in thermal or temporal properties.

118. The method of claim 116, wherein the first type of probe is a cleavable probe and the second type of probe is a non-cleavable probe.

119. The method of claim 118, wherein the first type of probe comprises a TaqMan probe, and the second type of probe comprises an extendable fluorogenic probe.

120. The method of claim 118, wherein the non-cleavable probe type is chosen from a universal extendable fluorogenic probe or an extendable hairpin probe designed for specific target amplification.

121. The method of claim 113, wherein the first stage of the cycle has a first temperature or temperature range and the second stage of the cycle has a second temperature or temperature range.

122. The method of claim 121, wherein the first temperature or temperature range does not overlap with the second temperature or temperature range.

123. The method of claim 121, wherein the first temperature or temperature range is higher than the second temperature or temperature range.

124. The method of claim 121, wherein the first temperature or temperature range is between about 80° C. and about 100° C.

125. The method of claim 121, wherein the second temperature or temperature range is between about 45° C. and about 75° C.

126. The method of claim 121, wherein the first stage of the cycle of the PCR assay is a denaturation stage and the second stage of the cycle of the PCR assay is one of an annealing stage, an extension stage or a combined annealing and extension stage.

127. The method of any one of claim 113, wherein the cycle of the PCR assay is an endpoint cycle of the PCR assay.

128. The method of claim 113, wherein the PCR assay is a digital PCR assay.

129. The method of claim 113, wherein the first subset of reaction sites corresponds to reaction sites of the plurality of reaction sites containing a first target analyte in the sample and the second subset of reaction sites corresponds to reaction sites of the plurality of reaction sites containing a second target analyte in the sample, the first and second target analytes differing from each other.

130. The method of claim 129, wherein the first subset of reaction sites and the second subset of reaction sites share at least some reaction sites in common.

131. The method of claim 129, wherein the second subset of reaction sites is a plurality of reaction sites of the first subset of reaction sites.

132. The method of claim 113, wherein the adjusted second emission signal data is generated by subtracting the subtracting the transformation value from the second emission signal data.
